(19) United States
(12) Patent Application Publication
(10) Pub. No.: US 2009/0092676 A1
(43) Pub. Date: Apr. 9, 2009

(54) CROSS-LINKED POLYMER PARTICLES

(75) Inventors: Robert E. Richard, Wrentham, MA (US); Goldi Kaul, Framingham, MA (US); John O'Gara, Ashland, MA (US); Robert G. Whirley, Santa Rosa, CA (US); Syed Askari, San Jose, CA (US)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 12/236,769

(22) Filed: Sep. 24, 2008

Related U.S. Application Data
(60) Provisional application No. 60/977,253, filed on Oct. 3, 2007.

(51) Int. Cl. A61K 9/14; C08L 81/00

(52) U.S. Cl. 424/501; 525/189

(57) ABSTRACT

Cross-linked polymer particles, as well as related compositions and methods, are disclosed.

### CROSS-REFERENCE TO RELATED APPLICATION

This application claims priority under 35 U.S.C. §119 to U.S. Ser. No. 60/977,253, filed Oct. 3, 2007, the contents of which are hereby incorporated by reference.

### TECHNICAL FIELD

The disclosure relates to cross-linked polymer particles, as well as related compositions and methods.

### BACKGROUND

Agents, such as therapeutic agents, can be delivered systemically, for example, by injection through the vascular system or oral ingestion, or they can be applied directly to a site where treatment is desired. In some cases, particles are used to deliver a therapeutic agent to a target site. Additionally or alternatively, particles may be used to perform embolization procedures and/or to perform radiotherapy procedures.

### SUMMARY

The invention relates to cross-linked polymers particles.

In one aspect, the invention features a particle including a cross-linked polymer network. The polymer network includes a moiety of Formula I:

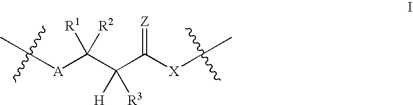

wherein:
A is S, NR⁴, or O;
X is CR⁶R⁷, O, or NR⁵;
Z is O or S;
R¹, R², and R³ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl;
R⁴ is H, alkyl, alkenyl, or alkynyl;
R⁵ is H, alkyl, alkenyl, or alkynyl;
or R¹ and R⁵ together with the atoms to which they are attached form a heterocycloalkyl ring, optionally substituted by 1, 2, or 3 substituents independently selected from halo, CN, NO₂, OH, alkoxy, haloalkoxy, amino, alkylamino, dialkylamino, alkyl, alkenyl, and alkynyl;
R⁶ and R⁷ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl; and
the particle has a maximum dimension of 5,000 microns or less.

In another aspect, the invention features a particle including a cross-linked polymer network. The cross-linked polymer network includes a moiety of Formula III:

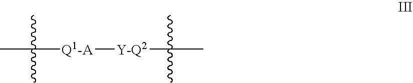

wherein:

A is S, NR⁴, or O;

Y is CR¹R²CHR³C(=Z)X;

X is CR⁶R⁷, O, or NR⁵;

Z is O or S;

R¹, R², and R³ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl;

R⁴ is H, alkyl, alkenyl, or alkynyl;

R⁵ is H, alkyl, alkenyl, or alkynyl;

or R¹ and R⁵ together with the atoms to which they are attached form a heterocycloalkyl ring, optionally substituted by 1, 2, or 3 substituents independently selected from halo, CN, NO₂, OH, alkoxy, haloalkoxy, amino, alkylamino, dialkylamino, alkyl, alkenyl, and alkynyl;

R⁶ and R⁷ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl;

Q¹ and Q² are independently selected from a polymer, a dendrimer, and a small molecule; and the particle has a maximum dimension of 5,000 microns or less.

In another aspect, the invention features a composition including a carrier fluid, a plurality of particles in the carrier fluid, where at least one particle includes a cross-linked polymer network including a moiety of Formula I.

In a further aspect, the invention features a method including delivering to a subject a composition that includes a substantially spherical polymer particle having a diameter of 5,000 microns or less. The particle includes a cross-linked polymer network including a moiety of Formula I.

In another aspect, the invention features a method of making a particle including: reacting a first reagent and a second reagent to form the particle, the first reagent includes at least two first reactive groups per molecule and a second reagent includes at least two second reactive groups per molecule. The particle has a maximum dimension of 5,000 microns or less.

In another aspect, the invention features a particle including a reaction product of a first and a second reagent. The first reagent includes at least two first reactive groups per molecule and a second reagent includes at least two second reactive groups per molecule. The particle has a maximum dimension of 5,000 microns or less.

In another aspect, the invention features a method of manufacturing a particle including selecting a desired particle compression force resistance, selecting a first reagent including at least two first reactive groups per molecule and a second reagent including at least two second reactive groups per molecule, selecting a mole ratio of the first reactive group and the second reactive group based on the desired particle compression force resistance, and reacting the first and second reagents at the selected mole ratio to form the particle. The particle has a maximum dimension of 5,000 microns or less.

In another aspect, the invention features a sponge including a cross-linked polymer network comprising a moiety of Formula I. The sponge is a hemostatic sponge.

In yet another aspect, the invention features a coil including a cross-linked polymer network comprising a moiety of Formula I. The coil is an embolic coil.

Embodiments may include one or more of the following features.

The particle can be resistant to a compression force of greater than or equal to 0.5 gram and less than or equal to 500 grams. In some embodiments, the desired particle compression force resistance can be greater than or equal to 0.5 gram and less than or equal to 500 grams. In some embodiments, the particle is an embolic particle. The particle can include pores.

In some embodiments, the particle further includes a therapeutic agent (e.g., a reactive therapeutic agent). The therapeutic agent can be covalently bonded to the polymer network, ionically bonded to the polymer network, and/or hydrogen bonded to the polymer network.

The polymer network can be crosslinked by a plurality of moieties having Formula I. The polymer network can include ethylene glycol monomer units. The polymer network can include poly(ethylene glycol) diacrylate. The polymer network can include olefinic monomer units, an acrylate, and/or an ionic charge. The polymer network can include one or more alkyl groups selected from 1,4-dimercapto-2,3-butanediol, pentaerythrithiol, and/or combinations thereof. The polymer network can form a gel.

In some embodiments, the cross-linked polymer network includes a moiety of Formula II:

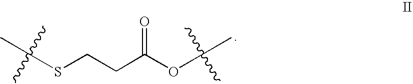

In some embodiments, the cross-linked polymer network includes a moiety of Formula IV:

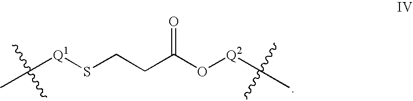

In some embodiments, A is S, Z is O, X is O, and/or R¹ and R³ together with the atoms to which they are attached form a succinimide. In some embodiments, Q¹ and Q² are each independently a polymer, a dendrimer, and/or an alkyl, optionally substituted with 1, 2, 3, 4, 5, or 6 substituents selected from halo, CN, NO₂, OH, alkoxy, haloalkoxy, amino, alkylamino, dialkylamino, alkyl, alkenyl, and/or alkynyl. In some embodiments, Q¹ is alkyl optionally substituted with 1 or 2 OH. In some embodiments, Q¹ is butyl 2,3-diol. In some embodiments, Q¹ is 3,3-diethylpentyl. In some embodiments, Q¹ is covalently bonded to two or more A. In some embodiments, Q² is a polymer, such as poly(ethylene glycol). Q² can be bonded to two or more Y.

The first reagent can be a crosslinking agent. The first reagent can include a polymer, a dendrimer, or a small molecule. The first reagent can include 1,4-dimercapto-2,3-butanediol, pentaerythrithiol, and/or combinations thereof The second reagent can include a polymer, a dendrimer, or a small molecule. The second reagent can include a polymer such as poly(ethylene glycol) diacrylate. In some embodiments, the mole ratio of the first reactive group and the second reactive group from 2:5 to 4:5.

The first reactive group can include thiols, amines, alcohols, and/or combinations thereof. The second reactive group can include moieties including reactive double bonds, such as acrylates, acrylamides, maleimides, vinyl sulfones, quinones, vinyl pyridinium, and/or combinations thereof The method can further include forming bonds between the reactive therapeutic agent, and the first and second reactive groups to form a crosslinked polymer. Forming bonds can include forming covalent bonds and/or ionic bonds. In some embodiments, the method includes forming covalent bonds and/or forming ionic bonds between the particle and a reactive therapeutic agent.

The carrier fluid can include a saline solution and/or a contrast agent.

Embodiments can include one or more of the following advantages.

The crosslinks can be biodegradable. For example, the reaction product that crosslinks polymer backbones can be biodegradable. This can be advantageous, for example, when it is desirable for the particle(s) to be absent from a body lumen after some desired time period (e.g., after the embolization is complete).

The particle can have a desired resistance to a compression force. In some embodiments, the desired compression force resistance can be tailored by selecting the mole ratio of the first and second reagents. As an example, a desired compression force resistance can be obtained by selecting a first reagent including at least two first reactive groups per molecule and a second reagent including at least two second reactive groups per molecule, selecting a mole ratio of the first reactive group and the second reactive group based on the desired compression force resistance, and reacting the first and second reagents at the selected mole ratio to form the particle.

One or more constituents of the particle can be chemically bound (e.g., bound via one or more covalent bonds, chelating bonds, ionic bonds, hydrogen bonds, van der Waals bonds, and/or electron donor-electron acceptor complexes) to one or more therapeutic agents. This can be advantageous, for example, when it is desirable to use the particle(s) to treat a disease (e.g., cancer, such as a cancerous tumor) using a therapeutic agent, alone or in combination with embolization. In some embodiments, an acrylate and/or a thiol functionality can covalently bond to one or more therapeutic agents. In some embodiments, a carbonyl or a thiol functionality in the particle can chelate to one or more therapeutic agents. Alternatively or in addition, the particle can include pores in which one or more therapeutic agents can be disposed.

The polymer backbones can be cross-linked at relatively low temperature and/or under relatively mild conditions. This can, for example, allow for one or more therapeutic agents to be combined with the polymers prior to cross-linking.

Features and advantages are in the description, drawings, and claims.

### DESCRIPTION OF DRAWINGS

FIG. 1 is a side view of an embodiment of a particle.

FIG. 2A depicts the material from which the particle shown in FIG. 1 is formed.

FIG. 2B depicts an embodiment of a precursor material.

FIG. 2C depicts an embodiment of a precursor material.

FIG. 2D depicts an embodiment of precursor materials.

FIG. 2E depicts an embodiment of a material from which a particle is formed.

FIGS. 3A-3C are an illustration of an embodiment of a system and method for producing particles.

FIG. 4A is a schematic illustrating the use of particles to embolize a lumen of a subject.

FIG. 4B is a greatly enlarged view of region 4B in FIG. 4A.

FIG. 5 is a cross-sectional view of an embodiment of a particle.

FIG. 6 is a cross-sectional view of an embodiment of a particle.

FIG. 7 is a side view of the proximal end portion of an embodiment of a device.

FIG. 8 is a side view of the distal end portion of the device of FIG. 7.

### DETAILED DESCRIPTION

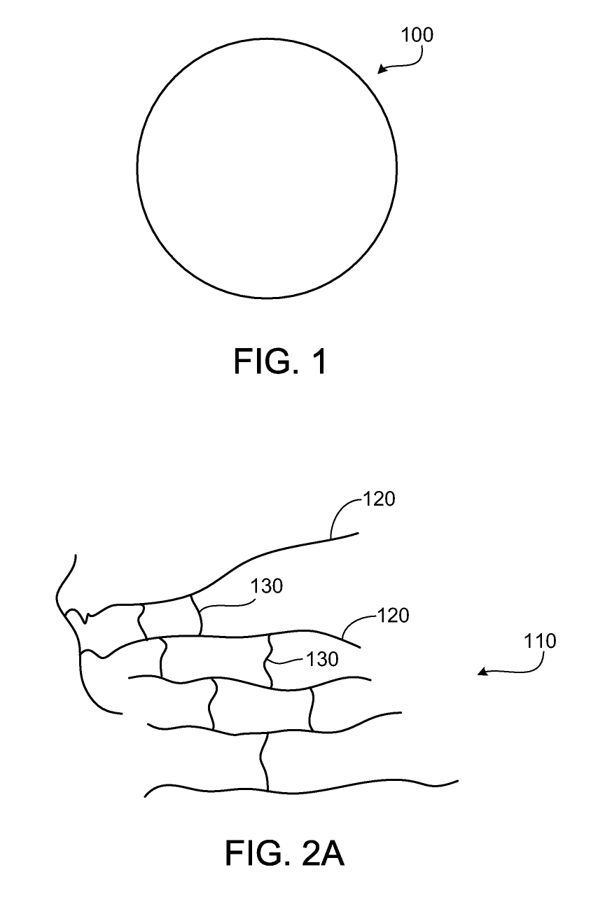

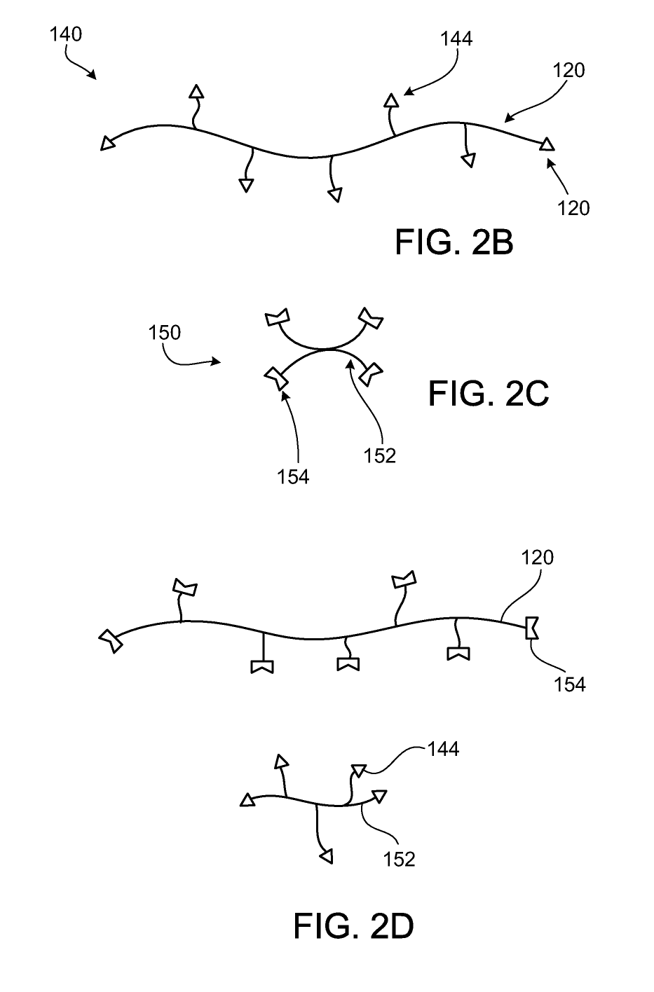

FIG. 1 shows a particle 100 that can be used, for example, in an embolization procedure. Particle 100 is formed of a crosslinked polymer network of a material 110, shown in FIG. 2A, that includes polymer backbones 120 and crosslinked moieties 130. FIGS. 2B and 2C depict a polymer 140 and a crosslinking agent 150, respectively, that are precursors to material 110. Polymer 140 includes a polymer backbone 120 and functionalities 144 covalently bonded to polymer backbone 120. Crosslinking agent 150 includes a spacer 152 and functionalities 154 covalently bonded to spacer 152. Crosslinked moieties 130 are formed by the reaction of functionalities 144 on polymer 140 and functionalities 154 on crosslinking agent 150.

In some embodiments, particle 100 can resist a compression force of greater than or equal to 0.5 gram (e.g., greater than or equal to one gram, greater than or equal to five grams, greater than or equal to 10 grams, greater than or equal to 15 grams, greater than or equal to 20 grams, greater than or equal to 30 grams, greater than or equal to 40 grams, greater than or equal to 50 grams, greater than or equal to 75 grams, greater than or equal to 100 grams, greater than or equal to 200 grams, greater than or equal to 300 grams, greater than or equal to 400 grams) and/or less than or equal to 500 grams, less than or equal to 400 grams, less than or equal to 300 grams, less than or equal to 200 grams, less than or equal to 100 grams, less than or equal to 75 grams, less than or equal to 50 grams, less than or equal to 40 grams, less than or equal to 30 grams, less than or equal to 20 grams, less than or equal to 15 grams, less than or equal to 10 grams, less than or equal to five grams, less than or equal to one gram). The compression force is measured by applying a force to the particle, while wet to 20 percent of its original diameter. The particle can regain a sphericity of 0.8 or more (e.g., 0.85 or more, 0.9 or more, or 1). The compression force resisted by a particle can be determined using a TA-texture Tech Compression Tester (Texture Technologies, Hamilton, Mass. 01982) at 80 percent strain. The compression force can be an average compression force of individually tested particles (e.g., 6 individually tested particles, 12 individually tested particles).

In some embodiments, the compression force that can be resisted by a particle is tailored by selecting the ratios of functionalities 154 to 144 in the particle. In some embodiments, the compression force that can be resisted by a particle depends on the volume of buffer solution used in particle synthesis. For example, a particle's resistance to compression force can be related to the ratio of functionalities 154 to 144 and/or the volume of a buffer solution via a mathematical relationship, such that the ratio of 154 to 144 for a desired particle resistance can be extrapolated from the relationship. For example, data can be analyzed using a software, such as a Design Expert (DE)™ software and critical variables controlling microsphere properties can be identified through analysis of variance (ANOVA). The software can pick a transformed model with a square root function to analyze the data. The analysis can afford an equation describing the dependence of compression force on the ratio of functionality 154 to functionality 144 and buffer volume. For example, the compression force can depend on the ratio of 154 to 144 and the buffer volume as shown by the following equation:

Sqrt(Compression Force)=−0.27662+[12.10421×(functionality 154)/(functionality 144)]−(1.93687×buffer volume)

In some embodiments, the polymer includes a polymer backbone that has multiple ethylene glycol monomer units. For example, the polymer can include polyethylene glycol. In some embodiments, the polymer backbone can include multiple aliphatic monomer units. The polymer can be linear or branched, and/or can include a mixture of two or more different monomer units, such that the polymer is a random copolymer, alternating copolymer, block copolymer, or graft copolymer. In some embodiments, the polymer is charged and contains negatively or positively charged ionic groups. The polymer can be biodegradable or non-biodegradable. Examples of polymers include poly(hydroxyl methacrylate)s (polyHEMAs), carbohydrates, polyacrylic acids, polymethacrylic acids, poly(vinyl sulfonate)s, carboxymethyl celluloses, hydroxyethyl celluloses, substituted celluloses, polyacrylamides, polyamides, polyureas, polyurethanes, polyesters, polyethers, polystyrenes, polysaccharides, polylactic acids, polyethylenes, polymethylmethacrylates, polycaprolactones, polyglycolic acids, poly(lactic-co-glycolic) acids (e.g., poly(d-lactic-co-glycolic) acids) and copolymers or mixtures thereof Polymers are described, for example, in Lanphere et al., U.S. Patent Application Publication No. US 2004/0096662 A1, published on May 20, 2004, and entitled “Embolization”; Song et al., U.S. patent application Ser. No. 11/314,056, filed on Dec. 21, 2005, and entitled “Block Copolymer Particles”; and Song et al., U.S. patent application Ser. No. 11/314,557, filed on Dec. 21, 2005, and entitled “Block Copolymer Particles”, all of which are incorporated herein by reference.

As shown in FIG. 2B, polymer backbone 120 can include two or more pendant functionalities 144, which can be located at the termini of the backbone, and/or attached to multiple locations branching from the polymer backbone. Functionalities 144 can include an activated double bonds having Formula IA:

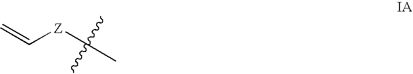

where Z is an electron withdrawing group, such as C═O, C(O)O, S═O, or SO₂. In some embodiments, functionalities 144 include α,β-unsaturated ketones and/or α,β-unsaturated esters. As an example, functionalities 144 can include acrylate, acrylamide, and/or vinylsulfone groups. In some embodiments, functionalities 144 are cyclic. For example, functionalities 144 can include maleimide, quinone, and/or vinylpyridinium groups. In some embodiments, the double bond in Formula IA is substituted with 1, 2, or 3 of the same or different substituents. For example, the substituents on the double bond can include H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and/or heterocycloalkylalkyl. In some embodiments, the alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and/or heterocycloalkylalkyl substituents are optionally substituted by 1, 2, or 3 of the same or different substituents such as halo, CN, NO₂, OH, alkoxy, haloalkoxy, amino, alkylamino, dialkylamino, alkyl, alkenyl, and/or alkynyl.

Referring again to FIG. 2C, spacer 152 can be a small molecule (e.g., a molecule having a molecular weight less than or equal to 1,000), an oligomer, or a polymer. Two or more functionalities 154 can be located at the termini of the spacer, or branch from multiple locations on the backbone of the spacer. The functionalities can include nucleophiles such as thiols, amines, hydroxyl groups, malonates, cyanoacetates, acetoacetates, and/or other β-keto esters. As an example, crosslinking agent 150 can include pentaerythrithiol and/or 1,4-dimercapto-2,3-butanediol. In some embodiments, crosslinking agent 150 is a dendrimer.

In some embodiments, functionalities 154 and/or 144 can be covalently bonded to a multifunctional (e.g., difunctional, trifunctional, etc.) oligomer. The oligomers can be biodegradable. Examples of biodegradable oligomers include low molecular weight polyethylene glycols, polysaccharides, polylactic acids, PGAs, polycaprolactones (e.g., poly-ε-caprolactone), polyglycolic acids, poly(lactic-co-glycolic) acids (e.g., poly(d-lactic-co-glycolic) acids, poly lactic acid (e.g., poly-L-lactic acid, poly-D,L-lactic acid), poly-p-dioxanones, poly(tri-methylene carbonate)s, polyanhydrides, poly(ortho ester)s, polyurethanes, poly(amino acid)s, poly(hydroxy alcanoate)s, polyphosphazenes, poly-b-malein acids, collagen (proteins), chitin, chitosan (polysaccharides), fibrin and albumin. In some embodiments, the oligomers are non-biodegradable. Examples of such oligomers include polyHEMAs, carbohydrates, polyacrylic acids, polymethacrylic acids, poly vinyl sulfonates, carboxymethyl celluloses, hydroxyethyl celluloses, substituted celluloses, polyacrylamides, polyamides, polyureas, polyurethanes, polyesters, polyethers, polystyrenes, polyethylenes, and polymethylmethacrylates. In some embodiments, the oligomers have low molecular weights, such that they can be excreted from the body.

Referring to FIG. 2D, while described above as being bonded to a polymer backbone or to a spacer on a crosslinking agent, in some embodiments, functionalities 144 and 154 can be interchanged such that functionalities 154 are bonded to the polymer backbone 120 and functionalities 144 are bonded to the crosslinking spacer 152.

Referring again to FIG. 2A, polymer 140 and crosslinking agent 150 can react via a Michael addition to form a polymer network of a material 110 which includes crosslinked moiety 130. Exemplary conditions for Michael additions are disclosed, for example, in Hubbell, J. et al., Biomacromolecules 2005, 6, 290-301; and International Patent Application Publication No. WO 00/44808. In some embodiments, referring to FIG. 2E, material 110 includes unreacted functionalities 144 and 154, which can further react with a reactive chemical species, such as a therapeutic agent. For example, in some embodiments, at most 10 percent (e.g., at most 20 percent, at most 40 percent, at most 60 percent, at most 80 percent, at most 100 percent) of the total amount of functionalities 144 and/or 154 in a polymer network are crosslinked.

In some embodiments, two or more types of polymer having one or more types of functionalities and/or two or more types of crosslinking agent having one or more types of functionalities are crosslinked together to form material 110.

In some embodiments, material 110 can include a multi-acrylated polyethylene glycol (e.g., poly(ethylene glycol) diacrylate) reacted with 1,4-dimercapto-2,3-butanediol, pentaerythrithiol, a dithiol, a tetrathiol, and/or a multi-thiol functionalized polyethylene glycol; a multi-vinyl sulfone functionalized polyethylene glycol reacted with a multi-thiol; a multi-maleimide functionalized polyethylene glycol reacted with a multi-amine; and/or a multi-thiol functionalized polyethylene glycol reacted with a multi-acrylate.

The crosslinked polymer network can be biodegradable and/or render a particle biodegradable when incorporated therein. As used herein, a biodegradable polymer is a polymer containing chemical linkages that can be broken down in the body by hydrolysis, enzymes and/or bacteria, to form a lower molecular weight species that can be absorbed by the body, or dissolved and be excreted by the body. For example, the polymer network can include ester groups, which can be hydrolyzed under physiological conditions to form lower molecular weight fragments.

In some embodiments, the crosslinked moiety can be biodegradable. A polymer network having a greater number of crosslinked moieties can degrade at a slower rate than a polymer network having a smaller number of crosslinked moieties.

The biodegradation can occur to a desirable extent in a time frame that can provide a therapeutic benefit. For example, the polymer network can have a mass reduction of about 10 percent or more, e.g. about 50 percent or more, after a period of one day or more within a body, e.g. about 60 days or more within a body, about 180 days or more within a body, about 600 days or more within a body, or 1000 days or less within a body.

In some embodiments, crosslinked polymer network 110 includes a moiety 130 having a structure of Formula I:

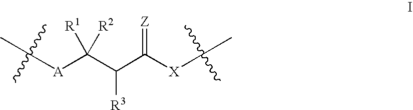

wherein: A is S, NR⁴, or O;

X is CR⁶R⁷, I, or NR⁵;

Z is O or S;

R¹, R², and R³ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl;

R⁴ is H, alkyl, alkenyl, or alkynyl;

R⁵ is H, alkyl, alkenyl, or alkynyl;

or R¹ and R⁵ together with the atoms to which they are attached form a heterocycloalkyl ring, optionally substituted by 1, 2, or 3 substituents independently selected from halo, CN, NO₂, OH, alkoxy, haloalkoxy, amino, alkylamino, dialkylamino, alkyl, alkenyl, and alkynyl; and

R⁶ and R⁷ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl.

In some embodiments, A is S.

In some embodiments, Z is O.

In some embodiments, X is O.

In some embodiments, R¹, R², and R³ are independently selected from H, halo, CN, NO₂, alkyl, alkenyl, alkynyl, haloalkyl, hydroxyalkyl, cyanoalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl.

In some embodiments, R¹, R², and R³ are independently selected from H, halo, CN, NO₂, alkyl, alkenyl, alkynyl, haloalkyl, hydroxyalkyl, and cyanoalkyl, wherein said -alkyl, alkenyl, alkynyl, haloalkyl, hydroxyalkyl, or cyanoalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl.

In some embodiments, R¹, R², and R³ are independently selected from H, halo, CN, NO₂, alkyl, alkenyl, and alkynyl, wherein said alkyl, alkenyl, or alkynyl is optionally substituted by 1, 2, or 3 substituents independently selected from halo, CN, NO₂, OH, alkoxy, haloalkoxy, amino, alkylamino, dialkylamino, alkyl, alkenyl, and alkynyl.

In some embodiments, R¹, R², and R³ are independently selected from H, halo, CN, NO₂, alkyl, alkenyl, and alkynyl.

In some embodiments, R¹, R², and R³ are independently selected from H, halo, CN, and NO₂.

In some embodiments, R¹, R², and R³ are independently H.

In some embodiments, R⁴ is H or alkyl.

In some embodiments, R⁴is H.

In some embodiments, R⁵ is H or alkyl.

In some embodiments, R⁵ is H.

In some embodiments, R¹ and R⁵ together with the atoms to which they are attached form a heterocycloalkyl ring, optionally substituted by 1, 2, or 3 substituents independently selected from halo, CN, NO₂, OH, alkoxy, amino, alkyl, alkenyl, and alkynyl.

In some embodiments, R¹ and R⁵ together with the atoms to which they are attached form a heterocycloalkyl ring, optionally substituted by 1, 2, or 3 substituents independently selected from halo, CN, NO₂, OH, alkoxy, and amino.

In some embodiments, R¹ and R⁵ together with the atoms to which they are attached form a succinimide.

In some embodiments, R⁶ and R⁷ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, and heteroarylalkyl, wherein said alkyl, alkenyl, alkynyl, haloalkyl, hydroxyalkyl, cyanoalkyl, aryl, heteroaryl, cycloalkyl, heterocycloalkyl, arylalkyl, or heteroarylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl.

In some embodiments, R⁶ and R⁷ are independently selected from H, halo, CN, NO₂, alkyl, alkenyl, alkynyl, haloalkyl, hydroxyalkyl, and cyanoalkyl, wherein said alkyl, alkenyl, alkynyl, haloalkyl, hydroxyalkyl, or cyanoalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl.

In some embodiments, R⁶ and R⁷ are independently selected from H, halo, CN, NO₂, alkyl, alkenyl, alkynyl, haloalkyl, hydroxyalkyl, and cyanoalkyl, wherein said alkyl, alkenyl, alkynyl, haloalkyl, hydroxyalkyl, or cyanoalkyl is optionally substituted by 1, 2, or 3 substituents independently selected from halo, CN, NO₂, OH, alkoxy, haloalkoxy, and amino.

In some embodiments, R⁶ and R⁷ are independently selected from H, halo, CN, NO₂, alkyl, alkenyl, and alkynyl, wherein said alkyl, alkenyl, or alkynyl is optionally substituted by 1, 2, or 3 substituents independently selected from halo, CN, NO₂, and OH.

In some embodiments, R⁶ and R⁷ are independently selected from H, halo, CN, NO₂, alkyl, alkenyl, and alkynyl.

In some embodiments, R⁶ and R⁷ are independently selected from H, halo, alkyl, alkenyl, and alkynyl.

In some embodiments, R⁶ and R⁷ are independently selected from H, halo, and alkyl.

In some embodiments, R⁶ and R⁷ are independently H.

In some embodiments, crosslinked moiety 130 includes a chemical structure of Formula II:

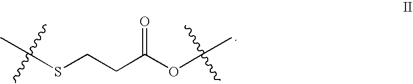

In some embodiments, a crosslinked polymer network 110 includes a structure of Formula III:

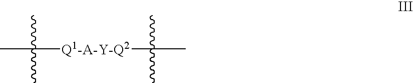

wherein:

A is S, NR⁴, or O;

Y is CR¹R²CHR³C(=Z)X;

X is CR⁶R⁷, O, or NR⁵;

Z is O or S;

R¹, R², and R³ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl;

R⁴ is H, alkyl, alkenyl, or alkynyl;

R⁵ is H, alkyl, alkenyl, or alkynyl;

or R¹ and R⁵ together with the atoms to which they are attached form a heterocycloalkyl ring, optionally substituted by 1, 2, or 3 substituents independently selected from halo, CN, NO₂, OH, alkoxy, haloalkoxy, amino, alkylamino, dialkylamino, alkyl, alkenyl, and alkynyl;

R⁶ and R⁷ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl; and

Q¹ and Q² are independently selected from a polymer, a dendrimer, and a small molecule.

In some embodiments, Q¹ and Q² are independently selected from a polymer, a dendrimer, and an alkyl, optionally substituted with 1, 2, 3, 4, 5, or 6 substituents selected from halo, CN, NO₂, OH, alkoxy, haloalkoxy, amino, alkylamino, dialkylamino, alkyl, alkenyl, and alkynyl.

In some embodiments, Q¹ is alkyl, optionally substituted with 1 or 2 OH.

In some embodiments, wherein Q¹ is butyl 2,3-diol.

In some embodiments, Q¹ is 3,3-diethylpentyl.

In some embodiments, Q¹ is covalently bonded to two or more A.

In some embodiments, Q² is a polymer.

In some embodiments, Q² is poly(ethylene glycol).

In some embodiments, Q² is bonded to two or more Y.

In some embodiments, crosslinked polymer network 110 comprises a moiety of Formula IV:

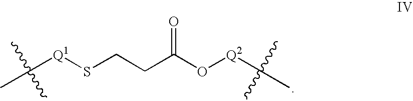

As used herein, the term “alkyl” refers to a saturated hydrocarbon group which is straight-chained or branched. Examples of alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to 20, from 2 to 20, from 1 to 10, from 1 to 8, from 1 to 6, from 1 to 4, or from 1 to 3 carbon atoms.

As used herein, “alkenyl” refers to an alkyl group having one or more double carbon-carbon bonds. Examples of alkenyl groups include ethenyl, propenyl, and the like. An alkenyl group can contain from 1 to 20, from 2 to 20, from 1 to 10, from 1 to 8, from 1 to 6, from 1 to 4, or from 1 to 3 carbon atoms.

As used herein, “alkynyl” refers to an alkyl group having one or more triple carbon-carbon bonds. Examples of alkynyl groups include ethynyl, propynyl, and the like. An alkynyl group can contain from 1 to 20, from 2 to 20, from 1 to 10, from 1 to 8, from 1 to 6, from 1 to 4, or from 1 to 3 carbon atoms.

As used herein, “haloalkyl” refers to an alkyl group having one or more halogen substituents. Examples of haloalkyl groups include CF₃, C₂F₅, CHF₂, CCl₃, CHCl₂, C₂Cl₅, and the like. A haloalkyl can contain from 1 to 20, from two to 20, from 1 to 10, from 1-8, from 1-6, from 1 to 4, or from 1 to 3 carbon atoms.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to 20 carbon atoms.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to 20 carbon atoms, 3 to 14 carbon atoms, 3 to 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of pentane, pentene, hexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like.

As used herein, a “heteroaryl” group refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to 20 carbon atoms, and in further embodiments from 3 to 20 carbon atoms. In some embodiments, the heteroaryl group contains 3 to 14, 3 to 7, or 5 to 6 ring-forming atoms. In some embodiments, the heteroaryl group has 1 to 4, 1 to 3, or 1 to 2 heteroatoms.

As used herein, “heterocycloalkyl” refers to a non-aromatic heterocycle where one or more of the ring-forming atoms is a heteroatom such as an O, N, or S atom. Heterocycloalkyl groups can include mono- or polycyclic (e.g., having 2, 3 or 4 fused rings) ring systems as well as spirocycles. Examples of “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. A heterocycloalkyl group having one or more fused aromatic rings can be attached though either the aromatic or non-aromatic portion. Also included in the definition of heterocycloalkyl are moieties in which any ring-forming C, N, or S atom bears one or two oxo substituents. In some embodiments, the heterocycloalkyl group has from 1 to 20 carbon atoms, and in further embodiments from 3 to 20 carbon atoms. In some embodiments, the heterocycloalkyl group contains 3 to 20, 3 to 14, 3 to 7, or 5 to 6 ring-forming atoms. In some embodiments, the heterocycloalkyl group has 1 to 4, 1 to 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “halo” or “halogen” includes fluoro, chloro, bromo, and iodo.

As used herein, “hydroxyalkyl” refers to an alkyl group substituted with a hydroxyl group. A hydroxyalkyl group can contain from 1 to 20, from 2 to 20, from 1 to 10, from 1 to 8, from 1 to 6, from 1 to 4, or from 1 to 3 carbon atoms.

As used herein, “cyanoalkyl” refers to an alkyl group substituted with a cyano group. A cyanoalkyl group can contain from 1 to 20, from 2 to 20, from 1 to 10, from 1 to 8, from 1 to 6, from 1 to 4, or from 1 to 3 carbon atoms.

As used herein, “alkoxy” refers to an —O-alkyl group. Examples of alkoxy groups include methoxy, ethoxy, propoxy (e.g., n-propoxy and isopropoxy), t-butoxy, and the like. An alkoxy group can contain from 1 to 20, from 2 to 20, from 1 to 10, from 1 to 8, from 1 to 6, from 1 to 4, or from 1 to 3 carbon atoms.

As used herein, “arylalkyl” refers to alkyl substituted by aryl and “cycloalkylalkyl” refers to alkyl substituted by cycloalkyl. An example of an arylalkyl group is benzyl.

As used herein, “heteroarylalkyl” refers to alkyl substituted by heteroaryl and “heterocycloalkylalkyl” refers to alkyl substituted by heterocycloalkyl.

As used herein, “amino” refers to NH₂.

As used herein, “alkylamino” refers to an amino group substituted by an alkyl group.

As used herein, “dialkylamino” refers to an amino group substituted by two alkyl groups.

In some embodiments, an alkyl, alkenyl, alkynyl, haloalkyl, hydroxylalkyl, cyanoalkyl, and/or alkoxy group can have greater than or equal to one carbon atom (e.g., greater than or equal to two, greater than or equal to three, greater than or equal to four, greater than or equal to five, greater than or equal to six, greater than or equal to seven, greater than or equal to eight, greater than or equal to nine, greater than or equal to 10, greater than or equal to 11, greater than or equal to 12, greater than or equal to 13, greater than or equal to 14, greater than or equal to 15, greater than or equal to 16, greater than or equal to 17, greater than or equal to 18, or greater than or equal to 19 carbon atoms) and/or less than or equal to 20 carbon atoms (e.g., less than or equal to 19, less than or equal to 18, less than or equal to 17, less than or equal to 16, less than or equal to 15, less than or equal to 14, less than or equal to 13, less than or equal to 12, less than or equal to 11, less than or equal to 10, less than or equal to nine, less than or equal to eight, less than or equal to seven, less than or equal to six, less than or equal to five, less than or equal to four, less than or equal to three, or less than or equal to two carbon atoms).

In some embodiments, an aryl group can have greater than or equal to six carbon atoms (e.g., greater than or equal to seven, greater than or equal to eight, greater than or equal to nine, greater than or equal to 10, greater than or equal to 11, greater than or equal to 12, greater than or equal to 13, greater than or equal to 14, greater than or equal to 15, greater than or equal to 16, greater than or equal to 17, greater than or equal to 18, or greater than or equal to 19 carbon atoms) and/or less than or equal to 20 carbon atoms (e.g., less than or equal to 19, less than or equal to 18, less than or equal to 17, less than or equal to 16, less than or equal to 15, less than or equal to 14, less than or equal to 13, less than or equal to 12, less than or equal to 11, less than or equal to 10, less than or equal to nine, less than or equal to eight, or less than or equal to seven carbon atoms).

In some embodiments, a cycloalkyl group can have greater than or equal to three carbon atoms (e.g., greater than or equal to four, greater than or equal to five, greater than or equal to six, greater than or equal to seven, greater than or equal to eight, greater than or equal to nine, greater than or equal to 10, greater than or equal to 11, greater than or equal to 12, greater than or equal to 13, greater than or equal to 14, greater than or equal to 15, greater than or equal to 16, greater than or equal to 17, greater than or equal to 18, or greater than or equal to 19 carbon atoms) and/or less than or equal to 20 carbon atoms (e.g., less than or equal to 19, less than or equal to 18, less than or equal to 17, less than or equal to 16, less than or equal to 15, less than or equal to 14, less than or equal to 13, less than or equal to 12, less than or equal to 11, less than or equal to 10, less than or equal to nine, less than or equal to eight, less than or equal to seven, less than or equal to six, less than or equal to five, or less than or equal to four carbon atoms).

In some embodiments, a heteroaryl and/or heterocycloalkyl group can have greater than or equal to three carbon atoms (e.g., greater than or equal to four, greater than or equal to five, greater than or equal to six, greater than or equal to seven, greater than or equal to eight, greater than or equal to nine, greater than or equal to 10, greater than or equal to 11, greater than or equal to 12, greater than or equal to 13, greater than or equal to 14, greater than or equal to 15, greater than or equal to 16, greater than or equal to 17, greater than or equal to 18, or greater than or equal to 19 carbon atoms) and/or less than or equal to 20 carbon atoms (e.g., less than or equal to 19, less than or equal to 18, less than or equal to 17, less than or equal to 16, less than or equal to 15, less than or equal to 14, less than or equal to 13, less than or equal to 12, less than or equal to 11, less than or equal to 10, less than or equal to nine, less than or equal to eight, less than or equal to seven, less than or equal to six, less than or equal to five, or less than or equal to four carbon atoms).

The particles can be formed using any desired technique. For example, particles can be formed by water-in-oil emulsions. As another example, particles can be formed by using a droplet generator to form a stream of drops of the polymers in an aqueous solvent that serve as precursors to the material from which the particle will be formed, placing the stream of drops into a bath of an appropriate liquid (e.g., an oil), and then homogenizing the liquid/polymer to form the drops. Exemplary droplet generator systems and methods are described below. Alternatively or additionally, particles can be formed using other techniques, such as, for example, molding.

The particle can be formed using a number of desired methods. As an example, in an water-in-oil emulsion process, specific weights or volumes of reagents 140 and 150, and optionally a therapeutic agent are mixed together in an aqueous phase (e.g., a buffer from pH 5 to 9) to promote reaction of reagents 140 and 150. In some embodiments, the buffer can be alkaline and have a pH from 7 to 9. Examples of buffers include glycine-glycine buffer, HEPES, tris glycine, bicine, and/or tricine buffers. Examples of oil phases include paraffin, mineral oil, olive oil, cyclohexane, palm oil, and/or corn oil. The aqueous mixture is added to an oil lipophilic phase including one or more emulsifiers to maintain a desired hydrophile-lipophile balance (HLB). The aqueous mixture is then emulsified by agitating and/or stirring into the lipophilic phase. The emulsion is stirred for an adequate amount of time (e.g., about 20 min, about 30 min, about 40 min, about 45 min, about 50 min, about 60 min, about 90 min, about 120 min, about 180 min, about 5 hours, about 10 hours, about 20 hours) to allow for reaction of reagents 140, 150, and/or therapeutic agent to form the particles. The particles are then washed with a suitable solvent to remove residual oil and emulsifier, and/or with water to remove any water-soluble impurities. Examples of suitable solvents include cyclohexane, ethers, chloroform, dichloromethane, benzene, and/or toluene.

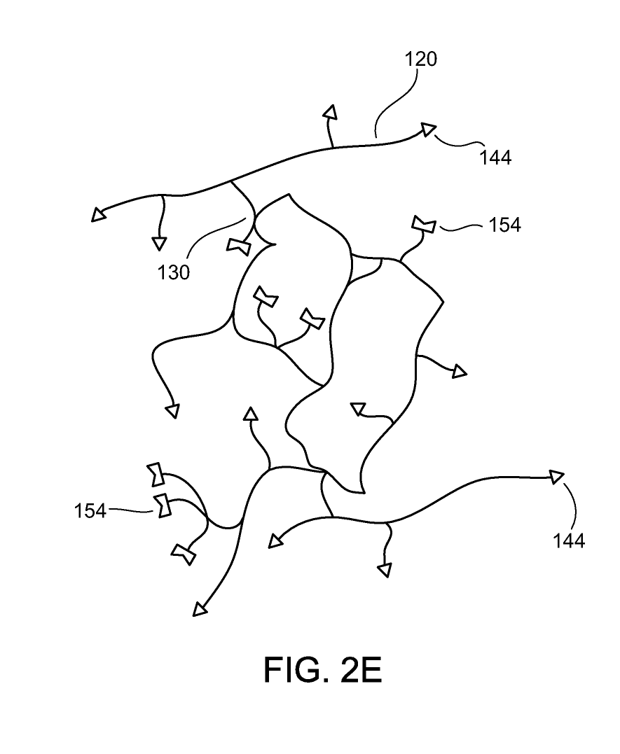

As another example, FIGS. 3A-3C show a single-emulsion process that can be used to make a particle. As shown in FIGS. 3A-3C, a drop generator 300 (e.g., a pipette, a needle) forms drops 310 of an aqueous solution including an aqueous solvent, a therapeutic agent, and reagents 140 and 150. Examples of aqueous solvents include water alone or in combination with suitable amounts of water-soluble organic solvents such as N,N-dimethylformamide (DMF), tetrahydrofuran (THF), N-methylpyrollidinone (NMP), and/or dimethylsulfoxide (DMSO). In certain embodiments, the organic solvent can be an aprotic polar solvent (e.g., DMF), which can dissolve both polar therapeutic agents and some non-polar therapeutic agents. In some embodiments, the aqueous solution includes an alkaline buffer such as glycine-glycine buffer, HEPES, tris glycine, bicine, and/or tricine buffers. In some embodiments, the aqueous solution can include at least five weight percent and/or at most 100 weight percent of the water. In this process, functionalities 154 and 144 can start reacting in the stream, and the reaction of these functionalities can be completed in the vessel.

After they are formed, drops 310 fall from drop generator 300 into a vessel 320 that contains an oil solution and an emulsifier or surfactant. Examples of emulsifiers or surfactants include lauryl sulfate, polyvinyl alcohols, poly(vinyl pyrrolidone) (PVP), and polysorbates (e.g., Tween® 20, Tween® 80), Span 80, and Span 40. The concentration of the emulsifier or surfactant in the oil phase can be at least 0. 1 percent w/v, and/or at most 20 percent w/v. For example, in some embodiments, the solution can include one percent w/v of Span 80 and Span 40.

As FIG. 3B shows, after drops 310 have fallen into vessel 320, the solution is mixed (e.g., homogenized) using a stirrer 330. In some embodiments, the solution can be mixed for a period of at least one minute and/or at most 24 hours. In certain embodiments, mixing can occur at a temperature of at least 10° C. and/or at most 100° C. The mixing results in a suspension 340 including particles 100 suspended in the oil phase (FIG. 3C).

After particles 100 have been formed, they are separated from the oil phase by, for example, filtration (e.g., through a drop funnel, filter paper, and/or a wire mesh), centrifuging followed by removal of the supernatant, and/or decanting. Thereafter, particles 100 are washed with an organic solvent (e.g., cyclohexane) to remove residual oil and emulsifier, and/or washed with water to remove unreacted monomer, and then either stored in water or alternately dried (e.g., by freeze drying, by evaporation, by vacuum drying, by air drying). If desired, the particles 100 can further be fractionated into different sizes using sieves and/or screens.

After particles 100 have been formed, they are separated from the solvent by, for example, filtration (e.g., through a drop funnel, filter paper, and/or a wire mesh), centrifugation followed by removal of the supernatant, and/or decantation. Thereafter, particles 100 are dried (e.g., by evaporation, by vacuum drying, by air drying).

In general, the maximum dimension of particle 100 is 5,000 microns or less (e.g., from two microns to 5,000 microns; from 10 microns to 5,000 microns; from 40 microns to 2,000 microns; from 100 microns to 700 microns; from 500 microns to 700 microns; from 100 microns to 500 microns; from 100 microns to 300 microns; from 300 microns to 500 microns; from 500 microns to 1,200 microns; from 500 microns to 700 microns; from 700 microns to 900 microns; from 900 microns to 1,200 microns; from 1,000 microns to 1,200 microns). In some embodiments, the maximum dimension of particle 100 is 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the maximum dimension of particle 100 is less than 100 microns (e.g., less than 50 microns).

In some embodiments, particle 100 can be substantially spherical. In certain embodiments, particle 100 can have a sphericity of 0.8 or more (e.g., 0.85 or more, 0.9 or more, 0.95 or more, 0.97 or more). Particle 100 can be, for example, manually compressed, essentially flattened, while wet to 50 percent or less of its original diameter and then, regain a sphericity of 0.8 or more (e.g., 0.85 or more, 0.9 or more, 0.95 or more, 0.97 or more, 1). The sphericity of a particle can be determined using a Beckman Coulter RapidVUE Image Analyzer version 2.06 (Beckman Coulter, Miami, Fla.). Briefly, the RapidVUE takes an image of continuous-tone (gray-scale) form and converts it to a digital form through the process of sampling and quantization. The system software identifies and measures particles in an image in the form of a fiber, rod or sphere. The sphericity of a particle, which is computed as Da/Dp (where Da=√(4A/π); Dp=P/π; A=pixel area; P=pixel perimeter), is a value from zero to one, with one representing a perfect circle.

In some embodiments, the particle's resistance to a compression force can be manipulated by the amount and type of porogen used during particle formation. Without wishing to be bound by theory, porogens can act to manipulate the amount of solid polymer per unit volume in the particle, i.e., alter the bulk density of the particle. A greater amount of solid polymer per unit volume can increase the particle's resistance to an external compressive force (e.g., decrease the compressibility), and a lesser amount of solid polymer per unit volume can decrease the particle's resistance to an external compressive force (e.g., increase the compressibility). Depending on the desired pore structure and particle compressibility, different types of porogens can be used. Without wishing to be bound by theory, in some embodiments, porogens can be water soluble and can act as a solvent for the monomers but as a non-solvent for the resulting polymer. The porogens can form pores having a maximum dimension less than 2 nm, pores having a maximum dimension between 2 nm and 50 nm, pores having a maximum dimension of greater than 50 nm, and/or cavities having a maximum dimension of at least 300 nm. In some embodiments, the porogen can be sparingly soluble in water and can form one or more discrete droplets within the aqueous monomer droplet, which can lead to the formation of discrete pores having a maximum dimension of greater than 50 nm and/or cavities having a maximum dimension of at least 300 nm in the particles, once the porogens are removed upon evaporation or dissolution. Examples of porogens include alkanes (e.g., isooctane, heptane, hexane, pentane), cycloalkanes having 5-12 carbon atoms (e.g., cyclohexane and methylcyclohexane), aromatic hydrocarbons (e.g., toluene, xylene, and benzene), volatile silicones (e.g., hexamethyldisiloxane, decamethyltetrasiloxane), C₄-C₁₅ alcohols (e.g., 4-methyl-2-pentanol), esters (e.g., butyl acetate), ethers having boiling points of less than about 180° C. (e.g., dibutyl ether), neutral surfactants, ionic surfactants, polyalkylene glycols, sugars, and/or air.

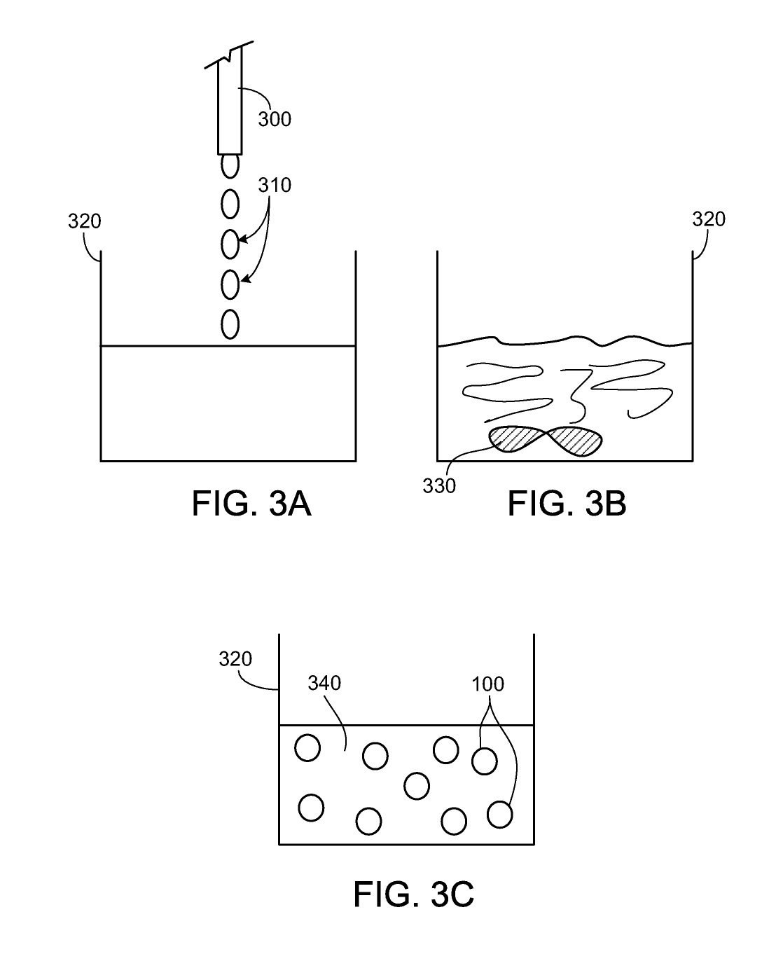

Multiple particles can be combined with a carrier fluid (e.g., a pharmaceutically acceptable carrier, such as a saline solution, a contrast agent, or both) to form a composition, which can then be delivered to a site and used to embolize the site. FIGS. 4A and 4B illustrate the use of a composition including particles to embolize a lumen of a subject. As shown, a composition including particles 100 and a carrier fluid is injected into a vessel through an instrument such as a catheter 450. Catheter 450 is connected to a syringe barrel 410 with a plunger 460. Catheter 450 is inserted, for example, into a femoral artery 420 of a subject. Catheter 450 delivers the composition to, for example, occlude a uterine artery 430 leading to a fibroid 440 located in the uterus of a female subject. The composition is initially loaded into syringe 410. Plunger 460 of syringe 410 is then compressed to deliver the composition through catheter 450 into a lumen 465 of uterine artery 430.

FIG. 4B, which is an enlarged view of section 4B of FIG. 4A, shows uterine artery 430, which is subdivided into smaller uterine vessels 470 (e.g., having a diameter of two millimeters or less) that feed fibroid 440. The particles 100 in the composition partially or totally fill the lumen of uterine artery 430, either partially or completely occluding the lumen of the uterine artery 430 that feeds uterine fibroid 440.

Compositions including particles such as particles 100 can be delivered to various sites in the body, including, for example, sites having cancerous lesions, such as the breast, prostate, lung, thyroid, or ovaries. The compositions can be used in, for example, neural, pulmonary, and/or AAA (abdominal aortic aneurysm) applications. The compositions can be used in the treatment of, for example, fibroids, tumors, internal bleeding, arteriovenous malformations (AVMs), and/or hypervascular tumors. The compositions can be used as, for example, fillers for aneurysm sacs, AAA sac (Type II endoleaks), endoleak sealants, arterial sealants, and/or puncture sealants, and/or can be used to provide occlusion of other lumens such as fallopian tubes. Fibroids can include uterine fibroids which grow within the uterine wall (intramural type), on the outside of the uterus (subserosal type), inside the uterine cavity (submucosal type), between the layers of broad ligament supporting the uterus (interligamentous type), attached to another organ (parasitic type), or on a mushroom-like stalk (pedunculated type). Internal bleeding includes gastrointestinal, urinary, renal and varicose bleeding. AVMs are, for example, abnormal collections of blood vessels (e.g. in the brain) which shunt blood from a high pressure artery to a low pressure vein, resulting in hypoxia and malnutrition of those regions from which the blood is diverted. In some embodiments, a composition containing the particles can be used to prophylactically treat a condition.

The magnitude of a dose of a composition can vary based on the nature, location and severity of the condition to be treated, as well as the route of administration. A physician treating the condition, disease or disorder can determine an effective amount of composition. An effective amount of embolic composition refers to the amount sufficient to result in amelioration of symptoms and/or a prolongation of survival of the subject, or the amount sufficient to prophylactically treat a subject. The compositions can be administered as pharmaceutically acceptable compositions to a subject in any therapeutically acceptable dosage, including those administered to a subject intravenously, subcutaneously, percutaneously, intratrachealy, intramuscularly, intramucosaly, intracutaneously, intra-articularly, orally or parenterally.

A composition can include a mixture of different particles, or can include particles that are all of the same type. In some embodiments, a composition can be prepared with a calibrated concentration of particles for ease of delivery by a physician. A physician can select a composition of a particular concentration based on, for example, the type of procedure to be performed. In certain embodiments, a physician can use a composition with a relatively high concentration of particles during one part of an embolization procedure, and a composition with a relatively low concentration of particles during another part of the embolization procedure.

Suspensions of particles in saline solution can be prepared to remain stable (e.g., to remain suspended in solution and not settle and/or float) over a desired period of time. A suspension of particles can be stable, for example, for from one minute to 20 minutes (e.g. from one minute to 10 minutes, from two minutes to seven minutes, from three minutes to six minutes).

In some embodiments, particles can be suspended in a physiological solution by matching the density of the solution to the density of the particles. In certain embodiments, the particles and/or the physiological solution can have a density of from one gram per cubic centimeter to 1.5 grams per cubic centimeter (e.g., from 1.2 grams per cubic centimeter to 1.4 grams per cubic centimeter, from 1.2 grams per cubic centimeter to 1.3 grams per cubic centimeter).

In certain embodiments, the carrier fluid of a composition can include a surfactant. The surfactant can help the particles to mix evenly in the carrier fluid and/or can decrease the likelihood of the occlusion of a delivery device (e.g., a catheter) by the particles. In certain embodiments, the surfactant can enhance delivery of the composition (e.g., by enhancing the wetting properties of the particles and facilitating the passage of the particles through a delivery device). In some embodiments, the surfactant can decrease the occurrence of air entrapment by the particles in a composition (e.g., by porous particles in a composition). Examples of liquid surfactants include Tween® 80 (available from Sigma-Aldrich) and Cremophor EL® (available from Sigma-Aldrich). An example of a powder surfactant is Pluronic® F127 NF (available from BASF). In certain embodiments, a composition can include from 0.05 percent by weight to one percent by weight (e.g., 0.1 percent by weight, 0.5 percent by weight) of a surfactant. A surfactant can be added to the carrier fluid prior to mixing with the particles and/or can be added to the particles prior to mixing with the carrier fluid.

In some embodiments, among the particles delivered to a subject (e.g., in a composition), the majority (e.g., 50 percent or more, 60 percent or more, 70 percent or more, 80 percent or more, 90 percent or more) of the particles can have a maximum dimension of 5,000 microns or less (e.g., 4,500 microns or less; 4,000 microns or less; 3,500 microns or less; 3,000 microns or less; 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, among the particles delivered to a subject, the majority of the particles can have a maximum dimension of less than 100 microns (e.g., less than 50 microns).

In certain embodiments, the particles delivered to a subject (e.g., in a composition) can have an arithmetic mean maximum dimension of 5,000 microns or less (e.g., 4,500 microns or less; 4,000 microns or less; 3,500 microns or less; 3,000 microns or less; 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the particles delivered to a subject can have an arithmetic mean maximum dimension of less than 100 microns (e.g., less than 50 microns).

Exemplary ranges for the arithmetic mean maximum dimension of particles delivered to a subject include from 100 microns to 500 microns; from 100 microns to 300 microns; from 300 microns to 500 microns; from 500 microns to 700 microns; from 700 microns to 900 microns; from 900 microns to 1,200 microns; and from 1,000 microns to 1,200 microns. In general, the particles delivered to a subject (e.g., in a composition) can have an arithmetic mean maximum dimension in approximately the middle of the range of the diameters of the individual particles, and a variance of 20 percent or less (e.g. 15 percent or less, 10 percent or less).

In some embodiments, the arithmetic mean maximum dimension of the particles delivered to a subject (e.g., in a composition) can vary depending upon the particular condition to be treated. As an example, in certain embodiments in which the particles are used to embolize a liver tumor, the particles delivered to the subject can have an arithmetic mean maximum dimension of 500 microns or less (e.g., from 100 microns to 300 microns; from 300 microns to 500 microns). As another example, in some embodiments in which the particles are used to embolize a uterine fibroid, the particles delivered to the subject can have an arithmetic mean maximum dimension of 1,200 microns or less (e.g., from 500 microns to 700 microns; from 700 microns to 900 microns; from 900 microns to 1,200 microns). As an additional example, in certain embodiments in which the particles are used to treat a neural condition (e.g., a brain tumor) and/or head trauma (e.g., bleeding in the head), the particles delivered to the subject can have an arithmetic mean maximum dimension of less than 100 microns (e.g., less than 50 microns). As a further example, in some embodiments in which the particles are used to treat a lung condition, the particles delivered to the subject can have an arithmetic mean maximum dimension of less than 100 microns (e.g., less than 50 microns). As another example, in certain embodiments in which the particles are used to treat thyroid cancer, the particles can have an arithmetic maximum dimension of 1,200 microns or less (e.g., from 1,000 microns to 1,200 microns). As an additional example, in some embodiments in which the particles are used only for therapeutic agent delivery, the particles can have an arithmetic mean maximum dimension of less than 100 microns (e.g., less than 50 microns, less than 10 microns, less than five microns).

The arithmetic mean maximum dimension of a group of particles can be determined using a Beckman Coulter RapidVUE Image Analyzer version 2.06 (Beckman Coulter, Miami, Fla.), described above. The arithmetic mean maximum dimension of a group of particles (e.g., in a composition) can be determined by dividing the sum of the diameters of all of the particles in the group by the number of particles in the group.

In some embodiments, particle 100 can have pores. For example, the polymer can form a matrix in which the pores are present by the addition of a porogen during particle synthesis. Additionally or alternatively, particle 100 can have one or more cavities. A pore can have a maximum dimension of at least 0.01 micron (e.g., at least 0.05 micron, at least 0.1 micron, at least 0.5 micron, at least one micron, at least two microns, at least five microns, at least 10 microns, at least 15 microns, at least 20 microns, at least 25 microns, at least 30 microns, at least 35 microns, at least 50 microns, at least 100 microns, at least 150 microns, at least 200 microns, or at least 250 microns), and/or at most 300 microns (e.g., at most 250 microns, at most 200 microns, at most 150 microns, at most 100 microns, at most 50 microns, at most 35 microns, at most 30 microns, at most 25 microns, at most 20 microns, at most 15 microns, at most 10 microns, at most five microns, at most two microns, at most one micron, at most 0.5 micron, at most 0.1 micron, or at most 0.05 micron). A cavity can have a maximum dimension of at least 300 microns (e.g., at least 500 microns, at least 750 microns, at least 1,000 microns, at least 2,000 microns, or at least 3,000 microns) and/or at most 4,000 microns (e.g., at most 3,000 microns, at most 2,000 microns, at most 1,000 microns, at most 750 microns, or at most 500 microns).

The presence of one or more cavities and/or one or more pores can enhance the ability of particle 100 to retain and/or deliver a relatively large volume of therapeutic agent. As an example, in some embodiments, a cavity can be used to store a relatively large volume of therapeutic agent, and/or pores can be used to deliver the relatively large volume of therapeutic agent into a target site within a body of a subject at a controlled rate. As another example, in certain embodiments, both a cavity and pores can be used to store and/or deliver one or more therapeutic agents. In some embodiments, a cavity can contain one type of therapeutic agent, while pores can contain a different type of therapeutic agent. As described above, particle 100 can be used to deliver one or more therapeutic agents (e.g., a combination of therapeutic agents) to a target site.

Therapeutic agents include genetic therapeutic agents, non-genetic therapeutic agents, and cells, and can be negatively charged, positively charged, amphoteric, or neutral. Therapeutic agents can be, for example, materials that are biologically active to treat physiological conditions; pharmaceutically active compounds; proteins; gene therapies; nucleic acids with and without carrier vectors (e.g., recombinant nucleic acids, DNA (e.g., naked DNA), cDNA, RNA, genomic DNA, cDNA or RNA in a non-infectious vector or in a viral vector which may have attached peptide targeting sequences, antisense nucleic acids (RNA, DNA)); oligonucleotides; gene/vector systems (e.g., anything that allows for the uptake and expression of nucleic acids); DNA chimeras (e.g., DNA chimeras which include gene sequences and encoding for ferry proteins such as membrane translocating sequences (“MTS”) and herpes simplex virus-1 (“VP22”)); compacting agents (e.g., DNA compacting agents); viruses; polymers; hyaluronic acid; proteins (e.g., enzymes such as ribozymes, asparaginase); immunologic species; nonsteroidal anti-inflammatory medications; oral contraceptives; progestins; gonadotrophin-releasing hormone agonists; chemotherapeutic agents; and radioactive species (e.g., radioisotopes, radioactive molecules). Examples of radioactive species include yttrium (⁹⁰Y), holmium (¹⁶⁶ Ho), phosphorus (³²P), (¹⁷⁷ Lu), actinium (²²⁵Ac), praseodymium, astatine (²¹¹At), rhenium (¹⁸⁶Re), bismuth (²¹²Bi or ²¹³Bi),), samarium (¹⁵³Sm), iridium (¹⁹²Ir), rhodium (¹⁰⁵Rh), iodine (¹³¹I, or ¹²⁵I), indium (¹¹¹In), technetium (⁹⁹Tc), phosphorus (³²P), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and/or gallium (⁶⁷Ga). In some embodiments, yttrium (⁹⁰Y), lutetium (¹⁷⁷ Lu), actinium (²²⁵Ac), praseodymium, astatine (²¹¹At), rhenium (¹⁶Re), bismuth (²¹²Bi or ²¹³Bi), holmium (¹⁶⁶Ho), samarium (¹⁵³Sm), iridium (¹⁹²Ir), and/or rhodium (¹⁰⁵Rh) can be used as therapeutic agents. In certain embodiments, yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), holmium (¹⁶⁶Ho), samarium (¹⁵³Sm), iridium (¹⁹²Ir), rhodium (105Rh), iodine (¹³¹I, or ¹²⁵I), indium (¹¹¹In), technetium (⁹⁹Tc), phosphorus (³²P), carbon (¹⁴C), and/or tritium (³H) can be used as a radioactive label (e.g., for use in diagnostics). In some embodiments, a radioactive species can be a radioactive molecule that includes antibodies containing one or more radioisotopes, for example, a radiolabeled antibody. Radioisotopes that can be bound to antibodies include, for example, iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹Tc), phosphorus (³²P), rhodium (¹⁰⁵Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and/or gallium (⁶⁷Ga). Examples of antibodies include monoclonal and polyclonal antibodies including RS7, Mov18, MN-14 IgG, CC49, COL-1, mAB A33, NP-4 F(ab′)2, anti-CEA, anti-PSMA, ChL6, m-170, or antibodies to CD20, CD74 or CD52 antigens. Examples of radioisotope/antibody pairs include m-170 MAB with ⁹⁰Y. Examples of commercially available radioisotope/antibody pairs include Zevalin™ (IDEC pharmaceuticals, San Diego, Calif.) and Bexxar™ (Corixa corporation, Seattle, Wash.). Further examples of radioisotope/antibody pairs can be found in J. Nucl. Med. 2003, April 44(4): 632-40.

Non-limiting examples of therapeutic agents include anti-thrombogenic agents; thrombogenic agents; agents that promote clotting; agents that inhibit clotting; antioxidants; angiogenic and anti-angiogenic agents and factors; anti-proliferative agents (e.g., agents capable of blocking smooth muscle cell proliferation, such as rapamycin); calcium entry blockers (e.g., verapamil, diltiazem, nifedipine); targeting factors (e.g., polysaccharides, carbohydrates); agents that can stick to the vasculature (e.g., charged moieties, such as gelatin, chitosan, and collagen); and survival genes which protect against cell death (e.g., anti-apoptotic Bcl-2 family factors and Akt kinase).

Examples of non-genetic therapeutic agents include: anti-thrombotic agents such as heparin, heparin derivatives, urokinase, and PPack (dextrophenylalanine proline arginine chloromethylketone); anti-inflammatory agents such as dexamethasone, prednisolone, corticosterone, budesonide, estrogen, acetyl salicylic acid, sulfasalazine and mesalamine; antineoplastic/antiproliferative/anti-mitotic agents such as paclitaxel, 5-fluorouracil, cisplatin, methotrexate, doxorubicin, vinblastine, vincristine, epothilones, endostatin, angiostatin, angiopeptin, monoclonal antibodies capable of blocking smooth muscle cell proliferation, and thymidine kinase inhibitors; anesthetic agents such as lidocaine, bupivacaine and ropivacaine; anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet factors or peptides; vascular cell growth promoters such as growth factors, transcriptional activators, and translational promoters; vascular cell growth inhibitors such as growth factor inhibitors (e.g., PDGF inhibitor-Trapidil), growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins, genistein, quinoxalines); prostacyclin analogs; cholesterol-lowering agents; angiopoietins; antimicrobial agents such as triclosan, cephalosporins, aminoglycosides and nitrofurantoin; cytotoxic agents, cytostatic agents and cell proliferation affectors; vasodilating agents; and agents that interfere with endogenous vasoactive mechanisms.

Examples of genetic therapeutic agents include: anti-sense DNA and RNA; DNA coding for anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, and insulin like growth factor, cell cycle inhibitors including CD inhibitors, thymidine kinase (“TK”) and other agents useful for interfering with cell proliferation, and the family of bone morphogenic proteins (“BMP's”), including BMP2, BMP3, BMP4, BMP5, BMP6 (Vgr1), BMP7 (OP1), BMP8, BMP9, BMP10, BM11, BMP12, BMP13, BMP14, BMP15, and BMP16. Currently preferred BMP's are any of BMP2, BMP3, BMP4, BMP5, BMP6 and BMP7. These dimeric proteins can be provided as homodimers, heterodimers, or combinations thereof, alone or together with other molecules. Alternatively or additionally, molecules capable of inducing an upstream or downstream effect of a BMP can be provided. Such molecules include any of the “hedgehog” proteins, or the DNA's encoding them. Vectors of interest for delivery of genetic therapeutic agents include: plasmids; viral vectors such as adenovirus (AV), adenoassociated virus (AAV) and lentivirus; and non-viral vectors such as lipids, liposomes and cationic lipids.

Cells include cells of human origin (autologous or allogeneic), including stem cells, or from an animal source (xenogeneic), which can be genetically engineered if desired to deliver proteins of interest.

Several of the above and numerous additional therapeutic agents are disclosed in Kunz et al., U.S. Pat. No. 5,733,925, which is incorporated herein by reference. Therapeutic agents disclosed in this patent include the following:

“Cytostatic agents” (i.e., agents that prevent or delay cell division in proliferating cells, for example, by inhibiting replication of DNA or by inhibiting spindle fiber formation). Representative examples of cytostatic agents include modified toxins, methotrexate, adriamycin, radionuclides (e.g., such as disclosed in Fritzberg et al., U.S. Pat. No. 4,897,255), protein kinase inhibitors, including staurosporin, a protein kinase C inhibitor of the following formula:

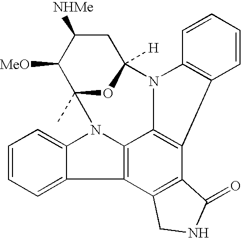

as well as diindoloalkaloids having one of the following general structures:

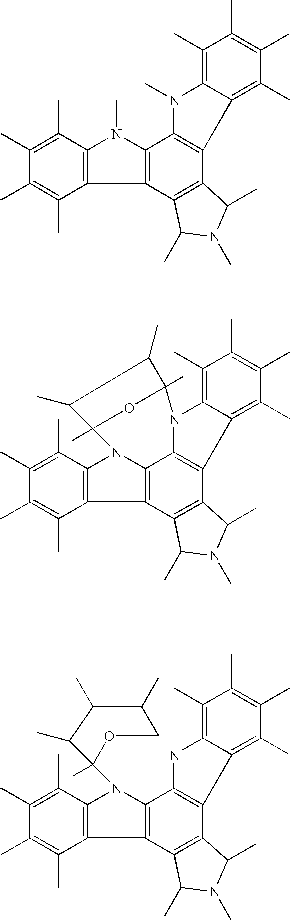

as well as stimulators of the production or activation of TGF-beta, including Tamoxifen and derivatives of functional equivalents (e.g., plasmin, heparin, compounds capable of reducing the level or inactivating the lipoprotein Lp(a) or the glycoprotein apolipoprotein(a)) thereof, TGF-beta or functional equivalents, derivatives or analogs thereof, suramin, nitric oxide releasing compounds (e.g., nitroglycerin) or analogs or functional equivalents thereof, paclitaxel or analogs thereof (e.g., taxotere), inhibitors of specific enzymes (such as the nuclear enzyme DNA topoisomerase II and DNA polymerase, RNA polymerase, adenyl guanyl cyclase), superoxide dismutase inhibitors, terminal deoxynucleotidyl-transferase, reverse transcriptase, antisense oligonucleotides that suppress smooth muscle cell proliferation and the like. Other examples of “cytostatic agents” include peptidic or mimetic inhibitors (i.e., antagonists, agonists, or competitive or non-competitive inhibitors) of cellular factors that may (e.g., in the presence of extracellular matrix) trigger proliferation of smooth muscle cells or pericytes: e.g., cytokines (e.g., interleukins such as IL-1), growth factors (e.g., PDGF, TGF-alpha or -beta, tumor necrosis factor, smooth muscle- and endothelial-derived growth factors, i.e., endothelin, FGF), homing receptors (e.g., for platelets or leukocytes), and extracellular matrix receptors (e.g., integrins). Representative examples of useful therapeutic agents in this category of cytostatic agents addressing smooth muscle proliferation include: subfragments of heparin, triazolopyrimidine (trapidil; a PDGF antagonist), lovastatin, and prostaglandins E1 or I2.

Agents that inhibit the intracellular increase in cell volume (i.e., the tissue volume occupied by a cell), such as cytoskeletal inhibitors or metabolic inhibitors. Representative examples of cytoskeletal inhibitors include colchicine, vinblastin, cytochalasins, paclitaxel and the like, which act on microtubule and microfilament networks within a cell. Representative examples of metabolic inhibitors include staurosporin, trichothecenes, and modified diphtheria and ricin toxins, Pseudomonas exotoxin and the like. Trichothecenes include simple trichothecenes (i.e., those that have only a central sesquiterpenoid structure) and macrocyclic trichothecenes (i.e., those that have an additional macrocyclic ring), e.g., a verrucarins or roridins, including Verrucarin A, Verrucarin B, Verrucarin J (Satratoxin C), Roridin A, Roridin C, Roridin D, Roridin E (Satratoxin D), Roridin H.

Agents acting as an inhibitor that blocks cellular protein synthesis and/or secretion or organization of extracellular matrix (i.e., an “anti-matrix agent”). Representative examples of “anti-matrix agents” include inhibitors (i.e., agonists and antagonists and competitive and non-competitive inhibitors) of matrix synthesis, secretion and assembly, organizational cross-linking (e.g., transglutaminases cross-linking collagen), and matrix remodeling (e.g., following wound healing). A representative example of a useful therapeutic agent in this category of anti-matrix agents is colchicine, an inhibitor of secretion of extracellular matrix. Another example is tamoxifen for which evidence exists regarding its capability to organize and/or stabilize as well as diminish smooth muscle cell proliferation following angioplasty. The organization or stabilization may stem from the blockage of vascular smooth muscle cell maturation in to a pathologically proliferating form.

Agents that are cytotoxic to cells, particularly cancer cells. Preferred agents are Roridin A, Pseudomonas exotoxin and the like or analogs or functional equivalents thereof. A plethora of such therapeutic agents, including radioisotopes and the like, have been identified and are known in the art. In addition, protocols for the identification of cytotoxic moieties are known and employed routinely in the art.

A number of the above therapeutic agents and several others have also been identified as candidates for vascular treatment regimens, for example, as agents targeting restenosis. Such agents include one or more of the following: calcium-channel blockers, including benzothiazapines (e.g., diltiazem, clentiazem); dihydropyridines (e.g., nifedipine, amlodipine, nicardapine); phenylalkylamines (e.g., verapamil); serotonin pathway modulators, including 5-HT antagonists (e.g., ketanserin, naftidrofuryl) and 5-HT uptake inhibitors (e.g., fluoxetine); cyclic nucleotide pathway agents, including phosphodiesterase inhibitors (e.g., cilostazole, dipyridamole), adenylate/guanylate cyclase stimulants (e.g., forskolin), and adenosine analogs; catecholamine modulators, including α-antagonists (e.g., prazosin, bunazosine), β-antagonists (e.g., propranolol), and α/β-antagonists (e.g., labetalol, carvedilol); endothelin receptor antagonists; nitric oxide donors/releasing molecules, including organic nitrates/nitrites (e.g., nitroglycerin, isosorbide dinitrate, amyl nitrite), inorganic nitroso compounds (e.g., sodium nitroprusside), sydnonimines (e.g., molsidomine, linsidomine), nonoates (e.g., diazenium diolates, NO adducts of alkanediamines), S-nitroso compounds, including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), C-nitroso-, O-nitroso- and N-nitroso-compounds, and L-arginine; ACE inhibitors (e.g., cilazapril, fosinopril, enalapril); ATII-receptor antagonists (e.g., saralasin, losartin); platelet adhesion inhibitors (e.g., albumin, polyethylene oxide); platelet aggregation inhibitors, including aspirin and thienopyridine (ticlopidine, clopidogrel) and GP Tib/IIIa inhibitors (e.g., abciximab, epitifibatide, tirofiban, intergrilin); coagulation pathway modulators, including heparinoids (e.g., heparin, low molecular weight heparin, dextran sulfate, β-cyclodextrin tetradecasulfate), thrombin inhibitors (e.g., hirudin, hirulog, PPACK (D-phe-L-propyl-L-arg-chloromethylketone), argatroban), Fxa inhibitors (e.g., antistatin, TAP (tick anticoagulant peptide)), vitamin K inhibitors (e.g., warfarin), and activated protein C; cyclooxygenase pathway inhibitors (e.g., aspirin, ibuprofen, flurbiprofen, indomethacin, sulfinpyrazone); natural and synthetic corticosteroids (e.g., dexamethasone, prednisolone, methprednisolone, hydrocortisone); lipoxygenase pathway inhibitors (e.g., nordihydroguairetic acid, caffeic acid; leukotriene receptor antagonists; antagonists of E- and P-selectins; inhibitors of VCAM-1 and ICAM-1 interactions; prostaglandins and analogs thereof, including prostaglandins such as PGE1 and PGI2; prostacyclins and prostacyclin analogs (e.g., ciprostene, epoprostenol, carbacyclin, iloprost, beraprost); macrophage activation preventers (e.g., bisphosphonates); HMG-CoA reductase inhibitors (e.g., lovastatin, pravastatin, fluvastatin, simvastatin, cerivastatin); fish oils and omega-3-fatty acids; free-radical scavengers/antioxidants (e.g., probucol, vitamins C and E, ebselen, retinoic acid (e.g., trans-retinoic acid), SOD mimics); agents affecting various growth factors including FGF pathway agents (e.g., bFGF antibodies, chimeric fusion proteins), PDGF receptor antagonists (e.g., trapidil), IGF pathway agents (e.g., somatostatin analogs such as angiopeptin and ocreotide), TGF-β pathway agents such as polyanionic agents (heparin, fucoidin), decorin, and TGF-β antibodies, EGF pathway agents (e.g., EGF antibodies, receptor antagonists, chimeric fusion proteins), TNF-α pathway agents (e.g., thalidomide and analogs thereof), thromboxane A2 (TXA2) pathway modulators (e.g., sulotroban, vapiprost, dazoxiben, ridogrel), protein tyrosine kinase inhibitors (e.g., tyrphostin, genistein, and quinoxaline derivatives); MMP pathway inhibitors (e.g., marimastat, ilomastat, metastat), and cell motility inhibitors (e.g., cytochalasin B); antiproliferative/antineoplastic agents including antimetabolites such as purine analogs (e.g., 6-mercaptopurine), pyrimidine analogs (e.g., cytarabine and 5-fluorouracil) and methotrexate, nitrogen mustards, alkyl sulfonates, ethylenimines, antibiotics (e.g., daunorubicin, doxorubicin, daunomycin, bleomycin, mitomycin, penicillins, cephalosporins, ciprofalxin, vancomycins, aminoglycosides, quinolones, polymyxins, erythromycins, tertacyclines, chloramphenicols, clindamycins, linomycins, sulfonamides, and their homologs, analogs, fragments, derivatives, and pharmaceutical salts), nitrosoureas (e.g., carmustine, lomustine) and cisplatin, agents affecting microtubule dynamics (e.g., vinblastine, vincristine, colchicine, paclitaxel, epothilone), caspase activators, proteasome inhibitors, angiogenesis inhibitors (e.g., endostatin, angiostatin and squalamine), and rapamycin, cerivastatin, flavopiridol and suramin; matrix deposition/organization pathway inhibitors (e.g., halofuginone or other quinazolinone derivatives, tranilast); endothelialization facilitators (e.g., VEGF and RGD peptide); and blood rheology modulators (e.g., pentoxifylline).

Other examples of therapeutic agents include anti-tumor agents, such as docetaxel, alkylating agents (e.g., mechlorethamine, chlorambucil, cyclophosphamide, melphalan, ifosfamide), plant alkaloids (e.g., etoposide), inorganics (e.g., cisplatin), biological response modifiers (e.g., interferon), and hormones (e.g., tamoxifen, flutamide), as well as their homologs, analogs, fragments, derivatives, and pharmaceutical salts.

Additional examples of therapeutic agents include organic-soluble therapeutic agents, such as mithramycin, cyclosporine, and plicamycin. Further examples of therapeutic agents include pharmaceutically active compounds, anti-sense genes, viral, liposomes and cationic polymers (e.g., selected based on the application), biologically active solutes (e.g., heparin), prostaglandins, prostcyclins, L-arginine, nitric oxide (NO) donors (e.g., lisidomine, molsidomine, NO-protein adducts, NO-polysaccharide adducts, polymeric or oligomeric NO adducts or chemical complexes), enoxaparin, Warafin sodium, dicumarol, interferons, interleukins, chymase inhibitors (e.g., Tranilast), ACE inhibitors (e.g., Enalapril), serotonin antagonists, 5-HT uptake inhibitors, and beta blockers, and other antitumor and/or chemotherapy drugs, such as BiCNU, busulfan, carboplatinum, cisplatinum, cytoxan, DTIC, fludarabine, mitoxantrone, velban, VP-16, herceptin, leustatin, navelbine, rituxan, and taxotere.

In some embodiments, a therapeutic agent can be hydrophilic. An example of a hydrophilic therapeutic agent is doxorubicin hydrochloride. In certain embodiments, a therapeutic agent can be hydrophobic. Examples of hydrophobic therapeutic agents include paclitaxel, cisplatin, tamoxifen, and doxorubicin base. In some embodiments, a therapeutic agent can be lipophilic. Examples of lipophilic therapeutic agents include taxane derivatives (e.g., paclitaxel) and steroidal materials (e.g., dexamethasone).

Therapeutic agents are described, for example, in DiMatteo et al., U.S. Patent Application Publication No. US 2004/0076582 A1, published on Apr. 22, 2004, and entitled “Agent Delivery Particle”; Schwarz et al., U.S. Pat. No. 6,368,658; Buiser et al., U.S. patent application Ser. No. 11/311,617, filed on Dec. 19, 2005, and entitled “Coils”; and Song, U.S. patent application Ser. No. 11/355,301, filed on Feb. 15, 2006, and entitled “Block Copolymer Particles”, all of which are incorporated herein by reference. In certain embodiments, in addition to or as an alternative to including therapeutic agents, particle 100 can include one or more radiopaque materials, materials that are visible by magnetic resonance imaging (MRI-visible materials), ferromagnetic materials, and/or contrast agents (e.g., ultrasound contrast agents). Radiopaque materials, MRI-visible materials, ferromagnetic materials, and contrast agents are described, for example, in Rioux et al., U.S. Patent Application Publication No. US 2004/0101564 A1, published on May 27, 2004, and entitled “Embolization”, which is incorporated herein by reference.

In some embodiments, the therapeutic agent is functionalized with a reactive group (e.g., functionalities 144 or 154) such that the therapeutic agent can be covalently bound to the polymer network. In some embodiments, material 110 can chelate to a therapeutic agent. For example, carbonyl groups can chelate to radioisotopes, such as Y⁹⁰. As an example, thiols can chelate to radioactive metal atoms.

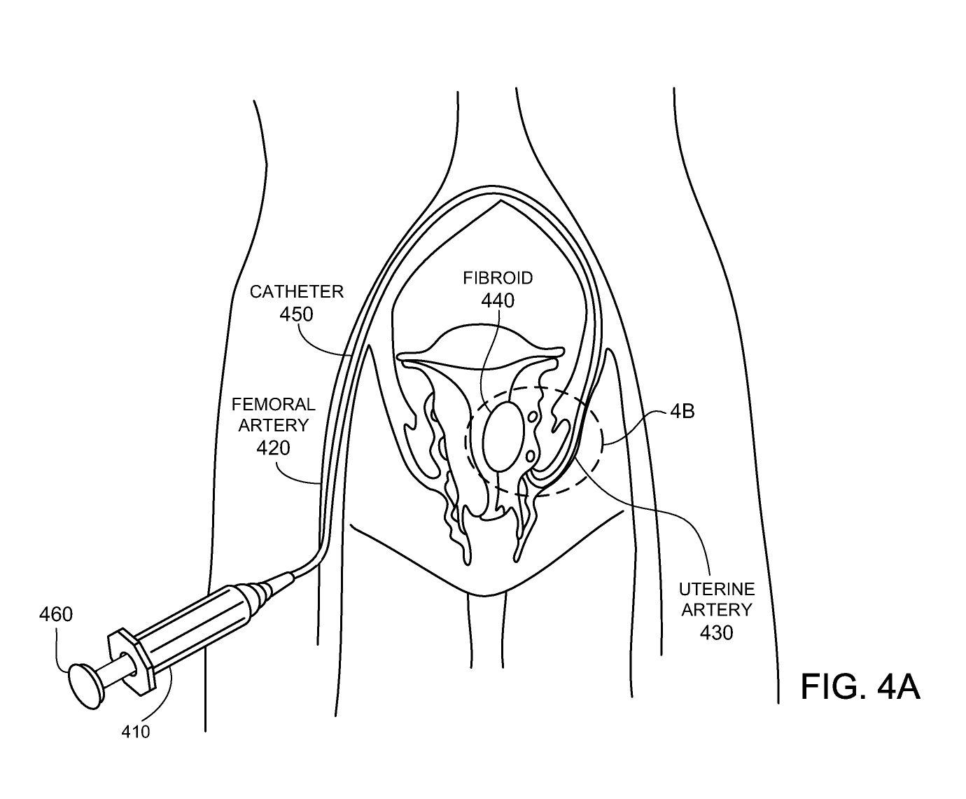

In certain embodiments, a particle can also include a coating. For example, FIG. 5 shows a particle 500 having an interior region 501 including a cavity 502 surrounded by a matrix 504. Matrix 504 includes pores 508, and is formed of material 110 described above. Particle 500 additionally includes a coating 510 formed of a polymer (e.g., alginate) that is different from the polymer in matrix 504. In some embodiments, the polymer coating includes the same polymer as in matrix 504 but at a different crosslinking ratio, such the material has different properties than the matrix. Coating 510 can, for example, regulate the release of therapeutic agent from particle 500, and/or provide protection to interior region 501 of particle 500 (e.g., during delivery of particle 500 to a target site). In certain embodiments, coating 510 can be formed of a bioerodible and/or bioabsorbable material that can erode and/or be absorbed as particle 500 is delivered to a target site. This can, for example, allow interior region 501 to deliver a therapeutic agent to the target site once particle 500 has reached the target site. A bioerodible material can be, for example, a polysaccharide (e.g., alginate); a polysaccharide derivative; an inorganic, ionic salt; a water soluble polymer (e.g., polyvinyl alcohol, such as polyvinyl alcohol that has not been cross-linked); biodegradable poly DL-lactide-poly ethylene glycol (PELA); a hydrogel (e.g., polyacrylic acid, hyaluronic acid, gelatin, carboxymethyl cellulose); a polyethylene glycol (PEG); chitosan; a polyester (e.g., a polycaprolactone); a poly(ortho ester); a polyanhydride; a poly(lactic-co-glycolic) acid (e.g., a poly(d-lactic-co-glycolic) acid); a poly(lactic acid) (PLA); a poly(glycolic acid) (PGA); or a combination thereof In some embodiments, coating 510 can be formed of a swellable material, such as a hydrogel (e.g., polyacrylamide co-acrylic acid). The swellable material can be made to swell by, for example, changes in pH, temperature, and/or salt. In certain embodiments in which particle 500 is used in an embolization procedure, coating 510 can swell at a target site, thereby enhancing occlusion of the target site by particle 500.

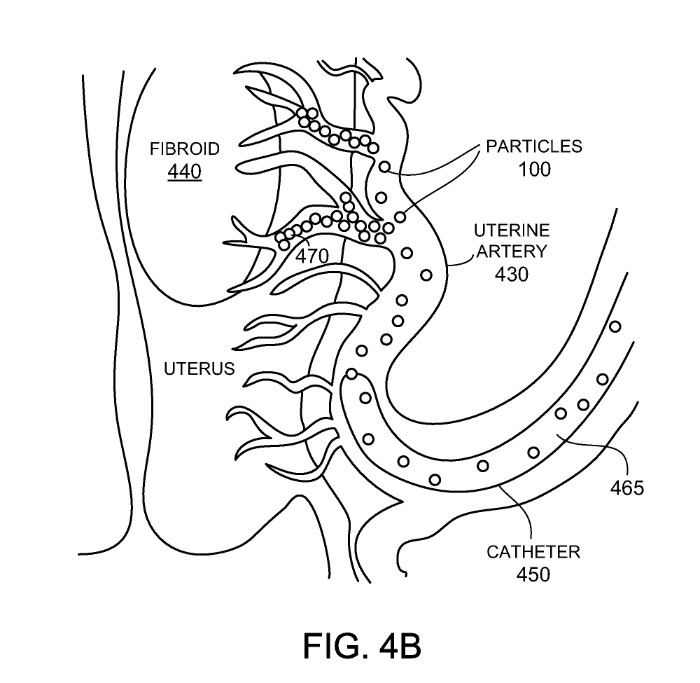

In some embodiments, a particle can include a porous coating that is formed of material 110 described above. For example, FIG. 6 shows a particle 600 including an interior region 602 and a coating 604. Coating 604 is formed of a matrix 606 that is formed of material 110 described above. Coating 604 also includes pores 608. In certain embodiments, interior region 602 can be formed of a swellable material. Pores 608 in coating 604 can expose interior region 602 to changes in, for example, pH, temperature, and/or salt. When interior region 602 is exposed to these changes, the swellable material in interior region 602 can swell, thereby causing particle 600 to become enlarged. In certain embodiments, coating 604 can be relatively flexible, and can accommodate the swelling of interior region 602. The enlargement of particle 600 can, for example, enhance occlusion during an embolization procedure.

Examples of swellable materials include hydrogels, such as polyacrylic acid, polyacrylamide co-acrylic acid, hyaluronic acid, gelatin, carboxymethyl cellulose, poly(ethylene oxide)-based polyurethane, polyaspartahydrazide, ethyleneglycoldiglycidylether (EGDGE), and polyvinyl alcohol (PVA) hydrogels. In some embodiments in which a particle includes a hydrogel, the hydrogel can be crosslinked, such that it may not dissolve when it swells. In other embodiments, the hydrogel may not be crosslinked, such that the hydrogel may dissolve when it swells.

In certain embodiments, a particle can include a coating that includes one or more therapeutic agents (e.g., a relatively high concentration of one or more therapeutic agents). One or more of the therapeutic agents can also be loaded into the interior region of the particle. Thus, the surface of the particle can release an initial dosage of therapeutic agent, after which the interior region of the particle can provide a burst release of therapeutic agent. The therapeutic agent on the surface of the particle can be the same as or different from the therapeutic agent in the interior region of the particle. The therapeutic agent on the surface of the particle can be applied to the particle by, for example, exposing the particle to a high concentration solution of the therapeutic agent.

In some embodiments, a therapeutic agent coated particle can include another coating over the surface of the therapeutic agent (e.g., a bioerodible polymer which erodes when the particle is administered). The coating can assist in controlling the rate at which therapeutic agent is released from the particle. For example, the coating can be in the form of a porous membrane. The coating can delay an initial burst of therapeutic agent release. In certain embodiments, the coating can be applied by dipping and/or spraying the particle. The bioerodible polymer can be a polysaccharide (e.g., alginate). In some embodiments, the coating can be an inorganic, ionic salt. Other examples of bioerodible coating materials include polysaccharide derivatives, water-soluble polymers (such as polyvinyl alcohol, e.g., that has not been cross-linked), biodegradable poly DL-lactide-poly ethylene glycol (PELA), hydrogels (e.g., polyacrylic acid, hyaluronic acid, gelatin, carboxymethyl cellulose), polyethylene glycols (PEG), chitosan, polyesters (e.g., polycaprolactones), poly(ortho esters), polyanhydrides, poly(lactic acids) (PLA), polyglycolic acids (PGA), poly(lactic-co-glycolic) acids (e.g., poly(d-lactic-co-glycolic) acids), and combinations thereof. The coating can include therapeutic agent or can be substantially free of therapeutic agent. The therapeutic agent in the coating can be the same as or different from an agent on a surface layer of the particle and/or within the particle. A polymer coating (e.g., a bioerodible coating) can be applied to the particle surface in embodiments in which a high concentration of therapeutic agent has not been applied to the particle surface. Coatings are described, for example, in DiMatteo et al., U.S. Patent Application Publication No. US 2004/0076582 A1, published on Apr. 22, 2004, and entitled “Agent Delivery Particle”, which is incorporated herein by reference.

The following examples are meant to be illustrative and not to be limiting.

### EXAMPLES

### Example 1

Materials: Pentaerythritol tetra bis (3-mercaptopropionate) (PETMP) MW=488 (Mfg: Bruno Bock Chemische Fabrik GmbH & Co, Germany), polyethylene glycol diacrylate (PEGDA) MW=742(Shearwater, Huntsville, Ala., USA), 0.4N Gly-Gly buffer-pH 7.91, paraffin oil (VWR International), sorbitan monooleate/Span 80 (TCI America) and sorbitan monopalmitate Span 40 (TCI America), cyclohexane (J. T. Baker), DI water, Normal saline.

Equipment: TA-texture Tech Compression Model TA.XT. Plus (Texture Technologies, Hamilton, Mass.), overhead stirrer e.g., RW11 basic overhead stirrer (IKA Works), 3 mL and 5 mL syringes (Becton-Dickinson), 3-way monomer resistant stopcocks (Qosina 99720, Quosina, N.Y.). Beckman Coulter RapidVUE Image Analyzer version 2.06 (Beckman Coulter, Miami, Fla.).

Microspheres were made using a water-in-oil (W/O) emulsification of the two or more reactants, e.g., a crosslinking agent and a polymer. The weight or volumes of the two reactants were mixed together in a 2-way syringe setup fitted with a 3-way stopcock, and the reaction was accelerated by addition of an alkaline buffer that deprotonates the nucleophilic thiol crosslinking agent. This mixture was then added to a paraffin oil phase containing a blend of emulsifiers (e.g., Spans) to maintain the required HLB. The aqueous phase solution was then emulsified under agitation/stirring using an overhead stirrer into the oily phase and allowed to stir for adequate time to allow for gelation of microspheres. The microspheres were then washed with an organic solvent such as cyclohexane to remove residual oil and emulsifier. The microspheres were also washed with distilled water to remove any water-soluble remnant monomer/impurity. The wet microspheres were then sieved for size with sieves, and fractions were collected in DI water or saline.

Specific reaction conditions for pentaerythritol tetra bis (3-mercaptopropionate) (PETMP) and polyethylene glycol diacrylate (PEGDA) are listed in Table 1. The product microspheres were nominally 500 μm in diameter (Beckman Coulter RapidVUE) and were characterized by compression force (TA-texture Tech Compression Model TA.XT) as described below. The data is listed in Table 1.

The relative amounts of Spans 80 and 40 were calculated as follows:

For a 0.5% w/v solution:

(4.3×Span 80)+(0.5−Span 80)×6.7=5

Span 80=0.35 g and so Span 40=(0.5−0.35)=0.15 g

For 1 % w/v solution:

(4.3×Span 80)+(1−Span 80)×6.7=5

Span 80=0.7 gandso Span40=0.3 g

For 2.0 % w/v solution:

(4.3×Span 80)+(2−Span 80)×6.7=5

Span 80=1.4 g and so Span 40=0.6 g

The compression force depended on the ratio of thiol to acrylate functionalities and the buffer volume as shown by the following equation:

Sqrt(Compression Force)=−0.27662+[12.10421×(functionality 154)/(functionality 144)]−(1.93687×buffer volume)

TABLE 1
Synthesis and Characterization Data

| | | | | | | | | Compression |
|---|---|---|---|---|---|---|---|---|
| | | | | | | Number | Mixing | Force (gm) |
| | | | Molar | | | of | Time in | of a 500 |
| | | | reactivity | pH | | mixing | Paraffin | micron |
| | | | ratio | 7.91 | Emulsifier | strokes | Oil | microsphere, |
| | PETMP | PEGDA | (thiol to | Buffer | concentration | for | (min) at | Ave (+SD) |
| Examples | (ml) | (ml) | acrylate) | (ml) | (% w/v) | reaction | 24 deg C. | N = 6 |
| 1A | 0.2 | 2 | 0.4 | 0.6 | 0.5 | 10 mixes | 45 | 1.63 (0.28) |
| | | | | | | in 10 sec | | |
| 1B | 0.2 | 2 | 0.4 | 1.5 | 2 | 10 mixes | 45 | 1.96 (0.54) |
| | | | | | | in 10 sec | | |
| 1C | 0.2 | 2 | 0.4 | 0.6 | 2 | 10 mixes | 45 | 11.92 (0.99) |
| | | | | | | in 10 sec | | |
| 1D | 0.4 | 2 | 0.8 | 1.5 | 2 | 10 mixes | 45 | 14.05 (5.3) |
| | | | | | | in 10 sec | | |
| 1E | 0.3 | 2 | 0.6 | 1.2 | 2 | 10 mixes | 45 | 18.56 (2.36) |
| | | | | | | in 10 sec | | |
| 1F | 0.2 | 2 | 0.4 | 1.2 | 1 | 10 mixes | 45 | 2.95 (0.72) |
| | | | | | | in 10 sec | | |
| 1G | 0.4 | 2 | 0.8 | 1.5 | 0.5 | 10 mixes | 45 | 23.46 (3.24) |
| | | | | | | in 10 sec | | |
| 1H | 0.3 | 2 | 0.6 | 1.5 | 1 | 10 mixes | 45 | 25.11 (4.66) |
| | | | | | | in 10 sec | | |
| 1I | 0.3 | 2 | 0.6 | 1.2 | 0.5 | 10 mixes | 45 | 25.96 (6.20) |
| | | | | | | in 10 sec | | |
| 1J | 0.3 | 2 | 0.6 | 1.2 | 1 | 10 mixes | 45 | 31.31 (4.88) |
| | | | | | | in 10 sec | | |
| 1K | 0.3 | 2 | 0.6 | 0.6 | 1 | 10 mixes | 45 | 33.25 (4.30) |
| | | | | | | in 10 sec | | |
| 1L | 0.3 | 2 | 0.6 | 1.2 | 1 | 10 mixes | 45 | 35.00 (3.90) |
| | | | | | | in 10 sec | | |
| 1M | 0.2 | 2 | 0.4 | 1.5 | 0.5 | 10 mixes | 45 | 4.28 (0.85) |
| | | | | | | in 10 sec | | |
| 1N | 0.3 | 2 | 0.6 | 1.2 | 1 | 10 mixes | 45 | 41.31 (3.92) |
| | | | | | | in 10 sec | | |
| 1O | 0.4 | 2 | 0.8 | 0.6 | 2 | 10 mixes | 45 | 49.64 (8.89) |
| | | | | | | in 10 sec | | |
| 1P | 0.4 | 2 | 0.8 | 1.2 | 1 | 10 mixes | 45 | 73.45 (5.04) |
| | | | | | | in 10 sec | | |
| 1Q | 0.4 | 2 | 0.8 | 0.6 | 0.5 | 10 mixes | 45 | 98.29 (8.14) |
| | | | | | | in 10 sec | | |

A sample calculation for molar reactivity ratio (thiol to acrylate) of 0.4 is given below:

1. Density of PEGDA=1.089 g/cc, Mol. Wt. of PEGDA=742

2. Density of PETMP=1.205 g/cc, Mol. Wt. of PETMP=488
For purposes of DOE, the quantity of PEGDA was kept a constant at 2 ml

2 ml of PEGDA=(1.089×2)=2.178 g or 0.002935 moles of PEGDA
For a molar reactivity ratio of thiol to acrylate=0.4

0.4=(4×moles of PETMP)/(0.002935 moles of PEGDA×2)
(as PETMP is a tetrafunctional and PEGDA a difunctional monomer)
Moles of PETMP=0.000587 moles
0.000587 moles of PETMP=0.286 g OR 0.34 ml
Therefore for a thiol/acrylate molar reactivity ratio of 0.4, 0.3 ml of PETMP was added to 2 ml of PEGDA.

The compression force/hardness and the compressibility of a particle can be determined using a TA-texture Tech Compression Model TA.XT. (Plus Texture Technologies, Hamilton, Mass. 01982) at 80 percent strain. The microspheres were tested for compression testing as follows:

The wet microspheres were placed on the 2″ diameter cylinder sample holder platform. Then, any excess water was carefully wicked away from an individual microsphere with a Texwipe ensuring that the sphere was not damaged or compressed in any manner. The sample cylinder with sphere was aligned beneath the 1.5 mm diameter cylindrical probe. The Elmo CCD camera, DVD player and TV monitor were all powered on.

Using a stereoscope at 10× to 20× magnification, while viewing through TV monitor, the focal plane was adjusted so that it viewed the top of the sphere surface. The probe was slowly nudged downwards onto the sphere till it just contacted the top of the sphere. A sample protocol was now run per a program so that the probe compressed and decompressed the microsphere three times at the rate of 0.1 mm/sec and compressed the sphere to 20% of its original size on every compression. The compression test was repeated for all 6 microspheres. The typical data output was a series of 3 “peaks” and “troughs” that corresponded to the compression force and decompression respectively. The apex of the first peak corresponded to the compression force in grams at 80% strain for the individual microsphere. The average gram force of the first peak for all the 6 microspheres gave the corresponding compression force reading in “gm” for that particular batch.

The compression recovery ratio of the microsphere was calculated as the ratio of the height of the third compression peak to the first compression peak. Cyclic stability of ˜1 indicates that the microsphere did not undergo any size or shape distortion on compression when tested in this manner. The recovery ratio range for sphericity was 0.80-1.00.

### Other Embodiments

While certain embodiments have been described, other embodiments are possible.

As an example, in some embodiments, enzymes and/or other bioactive agents can be mixed with the particles and/or co-injected with the particles (e.g. to facilitate degradation).

As another example, in some embodiments, particles can be used for tissue bulking. As an example, the particles can be placed (e.g., injected) into tissue adjacent to a body passageway. The particles can narrow the passageway, thereby providing bulk and allowing the tissue to constrict the passageway more easily. The particles can be placed in the tissue according to a number of different methods, for example, percutaneously, laparoscopically, and/or through a catheter. In certain embodiments, a cavity can be formed in the tissue, and the particles can be placed in the cavity. Particle tissue bulking can be used to treat, for example, intrinsic sphincteric deficiency (ISD), vesicoureteral reflux, gastroesophageal reflux disease (GERD), and/or vocal cord paralysis (e.g., to restore glottic competence in cases of paralytic dysphonia). In some embodiments, particle tissue bulking can be used to treat urinary incontinence and/or fecal incontinence. The particles can be used as a graft material or a filler to fill and/or to smooth out soft tissue defects, such as for reconstructive or cosmetic applications (e.g., surgery). Examples of soft tissue defect applications include cleft lips, scars (e.g., depressed scars from chicken pox or acne scars), indentations resulting from liposuction, wrinkles (e.g., glabella frown wrinkles), and soft tissue augmentation of thin lips. Tissue bulking is described, for example, in Bourne et al., U.S. Patent Application Publication No. US 2003/0233150 A1, published on Dec. 18, 2003, and entitled “Tissue Treatment”, which is incorporated herein by reference.

As an additional example, in certain embodiments, particles can be used to treat trauma and/or to fill wounds. In some embodiments, the particles can include one or more bactericidal agents and/or bacteriostatic agents.

As a further example, while compositions including particles suspended in at least one carrier fluid have been described, in certain embodiments, particles may not be suspended in any carrier fluid. For example, particles alone can be contained within a syringe, and can be injected from the syringe into tissue during a tissue ablation procedure and/or a tissue bulking procedure.

As an additional example, in some embodiments, particles having different shapes, sizes, physical properties, and/or chemical properties can be used together in a procedure (e.g., an embolization procedure). The different particles can be delivered into the body of a subject in a predetermined sequence or simultaneously. In certain embodiments, mixtures of different particles can be delivered using a multi-lumen catheter and/or syringe. In some embodiments, particles having different shapes and/or sizes can be capable of interacting synergistically (e.g., by engaging or interlocking) to form a well-packed occlusion, thereby enhancing embolization. Particles with different shapes, sizes, physical properties, and/or chemical properties, and methods of embolization using such particles are described, for example, in Bell et al., U.S. Patent Application Publication No. US 2004/0091543 A1, published on May 13, 2004, and entitled “Embolic Compositions”, and in DiCarlo et al., U.S. Patent Application Publication No. US 2005/0095428 A1, published on May 5, 2005, and entitled “Embolic Compositions”, both of which are incorporated herein by reference.

As a further example, in some embodiments in which a particle including a polymer is used for embolization, the particle can also include (e.g., encapsulate) one or more embolic agents, such as a sclerosing agent (e.g., ethanol), a liquid embolic agent (e.g., n-butyl-cyanoacrylate), and/or a fibrin agent. The other embolic agent(s) can enhance the restriction of blood flow at a target site.

As another example, while particles including a polymer have been described, in some embodiments, other types of medical devices and/or therapeutic agent delivery devices can include such a polymer. For example, in some embodiments, a coil can include a polymer as described above. In certain embodiments, the coil can be formed by flowing a stream of the polymer into an aqueous solution, and stopping the flow of the polymer stream once a coil of the desired length has been formed. Coils are described, for example, in Elliott et al., U.S. patent application Ser. No. 11/000,741, filed on Dec. 1, 2004, and entitled “Embolic Coils”, and in Buiser et al., U.S. patent application Ser. No. 11/311,617, filed on Dec. 19, 2005, and entitled “Coils”, both of which are incorporated herein by reference. In certain embodiments, sponges (e.g., for use as a hemostatic agent and/or in reducing trauma) can include a polymer as described above. In some embodiments, coils and/or sponges can be used as bulking agents and/or tissue support agents in reconstructive surgeries (e.g., to treat trauma and/or congenital defects).

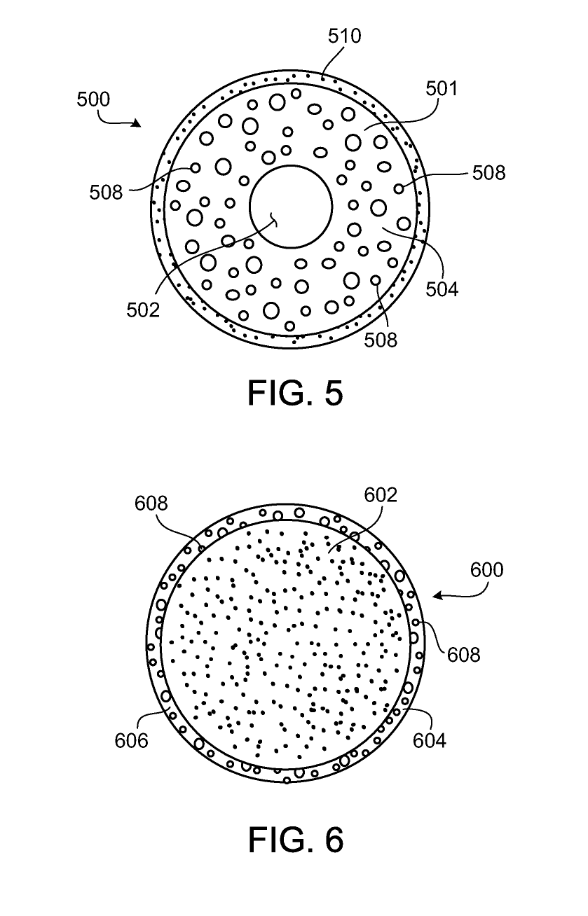

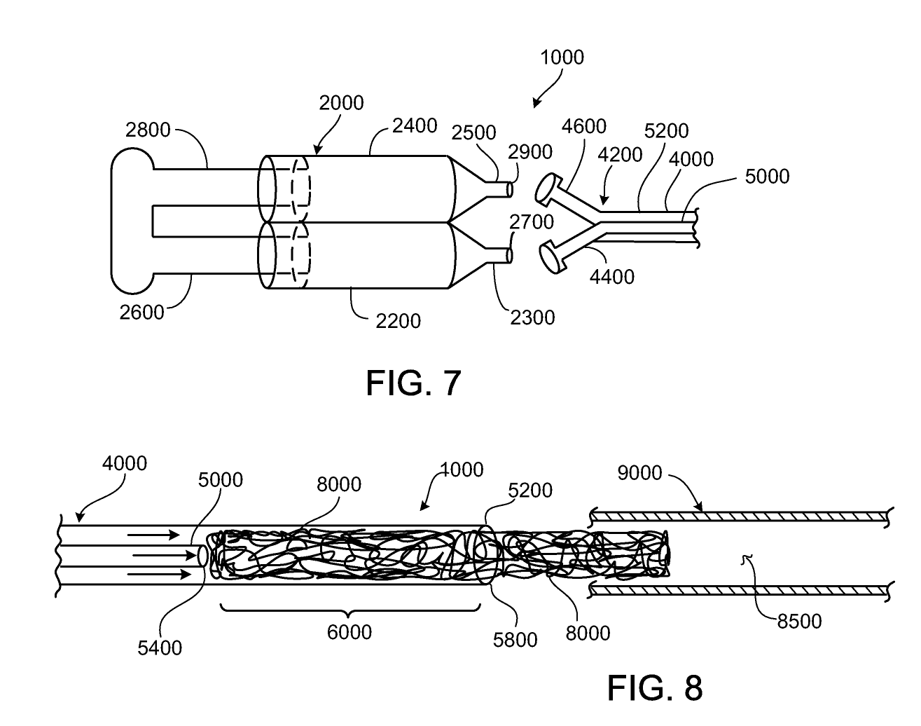

As a further example, in some embodiments, a treatment site can be occluded by using particles in conjunction with other occlusive devices. For example, particles can be used in conjunction with coils. Coils are described, for example, in Elliott et al., U.S. patent application Ser. No. 11/000,741, filed on Dec. 1, 2004, and entitled “Embolic Coils”, and in Buiser et al., U.S. patent application Ser. No. 11/311,617, filed on Dec. 19, 2005, and entitled “Coils”, both of which are incorporated herein by reference. In certain embodiments, particles can be used in conjunction with one or more gels. Gels are described, for example, in Richard et al., U.S. Patent Application Publication No. US 2006/0045900 A1, published on Mar. 2, 2006, and entitled “Embolization”, which is incorporated herein by reference. Additional examples of materials that can be used in conjunction with particles to treat a target site in a body of a subject include gel foams, glues, oils, and alcohol. Alternatively, or additionally, rather than using particles, a gel may be used. For example, as shown in FIGS. 7 and 8, a delivery device 1000 including a double-barrel syringe 2000 and a cannula 4000 that are capable of being coupled such that substances contained within syringe 2000 are introduced into cannula 4000. Syringe 2000 includes a first barrel 2200 having a tip 2300 with a discharge opening 2700, and a second barrel 2400 having a tip 2500 with a discharge opening 2900. Syringe 2000 further includes a first plunger 2600 that is movable in first barrel 2200, and a second plunger 2800 that is movable in second barrel 2400. As an example, first barrel 2200 can contain polymer 140, and second barrel 2400 can contain crosslinking agent 150. In its proximal end portion, cannula 4000 includes an adapter 4200 with a first branch 4400 that can connect with tip 2300, and a second branch 4600 that can connect with tip 2500. First branch 4400 is integral with a first tubular portion 5000 of cannula 4000, and second branch 4600 is integral with a second tubular portion 5200 of cannula 4000. First tubular portion 5000 is disposed within second tubular portion 5200. Delivery devices are described, for example, in Sahatjian et al., U.S. Pat. No. 6,629,947, which is incorporated herein by reference. When cannula 4000 is connected to syringe 2000 and plungers 2600 and 2800 are depressed, crosslinking agent 150 moves from second barrel 2400 into second tubular portion 5200, and polymer 140 moves from first barrel 2200 into first tubular portion 5000. Polymer 140 exits first tubular portion 5000 and contacts crosslinking agent 150 in a mixing section 6000 of second tubular portion 5200. Functionalities 144 and 154 react to form material 110 in the form of a gel (e.g., a biocompatible gel) 8000 within mixing section 6000. Gel 8000 exits delivery device 1000 at a distal end 5800 of mixing section 6000, and is delivered into a lumen 8500 of a vessel 9000 of a subject (e.g., an artery of a human) where gel 8000 can embolize lumen 8500 and/or deliver a therapeutic agent. In certain embodiments, gel 8000 is formed in lumen 8500 (e.g., when mixing section 6000 is in lumen 8500 when functionalities 144 and 154 react). In some embodiments, gel 8000 can be formed outside the body and subsequently delivered into lumen 8500.

Other embodiments are in the claims.

1. A particle, comprising:
a cross-linked polymer network comprising a moiety of Formula I: 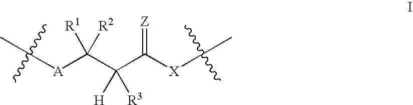
wherein:
A is S, NR⁴, or O;
X is CR⁶R⁷, O, or NR⁵;
Z is O or S;
R¹, R², and are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl;
R⁴ is H, alkyl, alkenyl, or alkynyl;
R⁵ is H, alkyl, alkenyl, or alkynyl;
or R¹ and R⁵ together with the atoms to which they are attached form a heterocycloalkyl ring, optionally substituted by 1, 2, or 3 substituents independently selected from halo, CN, NO₂, OH, alkoxy, haloalkoxy, amino, alkylamino, dialkylamino, alkyl, alkenyl, and alkynyl;
R⁶ and R⁷ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl; and
the particle has a maximum dimension of 5,000 microns or less, and the particle is resistant to a compression force of greater than or equal to 0.5 gram and less than or equal to 500 grams.

2. The particle of claim 1, wherein A is S.

3. The particle of claim 1, wherein Z is O.

4. The particle of claim 1, wherein X is O.

5. The particle of claim 1, wherein the cross-linked polymer network comprises a moiety of Formula II:

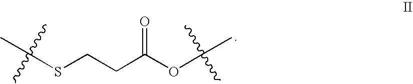

6. The particle of claim 1, wherein the polymer network comprises ethylene glycol monomer units.

7. The particle of claim 1, wherein the polymer network comprises poly(ethylene glycol) diacrylate.

8. The particle of claim 1, wherein the polymer network comprises olefinic monomer units.

9. The particle of claim 1, wherein the polymer network comprises one or more alkyl groups selected from 1,4-dimercapto-2,3-butanediol, pentaerythrithiol, and combinations thereof.

10. The particle of claim 1, wherein R¹ and R³ together with the atoms to which they are attached form a succinimide.

11. The particle of claim 1, wherein the polymer network is crosslinked by a plurality of moieties having Formula I.

12. The particle of claim 1, wherein the particle further comprises a therapeutic agent.

13. The particle of claim 12, wherein the therapeutic agent is covalently bonded to the polymer network.

14. The particle of claim 12, wherein the therapeutic agent is ionically bonded to the polymer network.

15. The particle of claim 12, wherein the therapeutic agent is hydrogen bonded to the polymer network.

16. The particle of claim 1, wherein the polymer network forms a gel.

17. The particle of claim 1, wherein the polymer network comprises an acrylate.

18. The particle of claim 1, wherein the polymer network comprises an ionic charge.

19. The particle of claim 1, wherein the particle is an embolic particle.

20. The particle of claim 1, wherein the particle includes pores.

21. A particle, comprising:
a cross-linked polymer network comprising a moiety of Formula III: 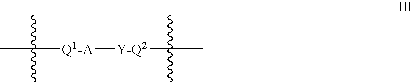
wherein:
A is S, NR⁴, or O;
Y is CR¹R²CHR³C(=Z)X;
X is CR⁶R⁷, O, or NR⁵;
Z is O or S;
R¹, R², and are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl;
R⁴ is H, alkyl, alkenyl, or alkynyl;
R⁵ is H, alkyl, alkenyl, or alkynyl;
or R¹ and R⁵ together with the atoms to which they are attached form a heterocycloalkyl ring, optionally substituted by 1, 2, or 3 substituents independently selected from halo, CN, NO₂, OH, alkoxy, haloalkoxy, amino, alkylamino, dialkylamino, alkyl, alkenyl, and alkynyl;
R⁶ and R⁷ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl;
Q¹ and Q² are independently selected from a polymer, a dendrimer, and a small molecule; and
the particle has a maximum dimension of 5,000 microns or less, and the particle is resistant to a compression force of greater than 0.5 gram and less than or equal to 500 grams..

22-33. (canceled)

34. A composition, comprising:
a carrier fluid,
a plurality of particles in the carrier fluid;
wherein at least one particle includes a cross-linked polymer network comprising a moiety of Formula I: 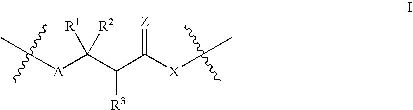
wherein:
A is S, NR⁴,or O;
X is CR⁶R⁷, O, or NR⁵;
Z is O or S;
R¹, R², and are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl;
R⁴ is H, alkyl, alkenyl, or alkynyl;
R⁵ is H, alkyl, alkenyl, or alkynyl;
or R¹ and R⁵ together with the atoms to which they are attached form a heterocycloalkyl ring, optionally substituted by 1, 2, or 3 substituents independently selected from halo, CN, NO₂, OH, alkoxy, haloalkoxy, amino, alkylamino, dialkylamino, alkyl, alkenyl, and alkynyl;
R⁶ and R⁷ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl; and
the particle has a maximum dimension of 5,000 microns or less, and the particle is resistant to a compression force of greater than 0.5 gram and less than or equal to 500 grams.

35.-54. (canceled)

55. A particle, comprising:
a reaction product of a first and a second reagent, wherein the first reagent includes at least two first reactive groups per molecule and a second reagent includes at least two second reactive groups per molecule, the particle has a maximum dimension of 5,000 microns or less, and the particle is resistant to a compression force of greater than 0.5 gram and less than or equal to 500 grams..

56-72. (canceled)

73. A sponge, comprising:
a cross-linked polymer network comprising a moiety of Formula I: 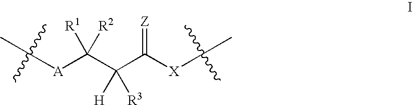
wherein:
A is S, NR⁴, or O;
X is CR⁶R⁷, O, or NR⁵;
Z is O or S;
R¹, R², R³ and are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl;
R⁴ is H, alkyl, alkenyl, or alkynyl;
R⁵ is H, alkyl, alkenyl, or alkynyl;
or R¹ and R⁵ together with the atoms to which they are attached form a heterocycloalkyl ring, optionally substituted by 1, 2, or 3 substituents independently selected from halo, CN, NO₂, OH, alkoxy, haloalkoxy, amino, alkylamino, dialkylamino, alkyl, alkenyl, and alkynyl;
R⁶ and R⁷ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl; and
the sponge is a hemostatic sponge.

74. A coil comprising:
a cross-linked polymer network comprising a moiety of Formula I: 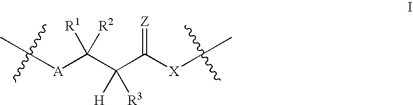
wherein:
A is S, NR⁴, or O;
X is CR⁶R⁷, O, or NR⁵;
Z is O or S;
R¹, R², R³ and are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl;
R⁴ is H, alkyl, alkenyl, or alkynyl;
R⁵ is H, alkyl, alkenyl, or alkynyl;
or R¹ and R⁵ together with the atoms to which they are attached form a heterocycloalkyl ring, optionally substituted by 1, 2, or 3 substituents independently selected from halo, CN, NO₂, OH, alkoxy, haloalkoxy, amino, alkylamino, dialkylamino, alkyl, alkenyl, and alkynyl;
R⁶ and R⁷ are independently selected from H, halo, CN, NO₂, alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, and heterocycloalkylalkyl, wherein said alkyl, alkenyl, alkynyl, haloalkyl, hydroxyalkyl, cyanoalkyl, aryl, heteroaryl, cycloalkyl, heterocycloalkyl, arylalkyl, heteroarylalkyl, cycloalkylalkyl, or heterocycloalkylalkyl is optionally substituted by 1, 2, or 3 substituents independently selected from halo, CN, NO₂, OH, alkoxy, haloalkoxy, amino, alkylamino, dialkylamino, alkyl, alkenyl, and alkynyl; and
the coil is an embolic coil.
